(19) United States
(12) Patent Application Publication
(10) Pub. No.: US 2024/0327794 A1
(43) Pub. Date: Oct. 3, 2024

(54) FOODSTUFFS COMPRISING CELLS DIFFERENTIATED FROM ENGINEERED OLIGOPOTENT STEM CELLS

(71) Applicant: SUPRÊME, Evry-Courcouronnes Cedex (FR)

(72) Inventors: Federico Jose GONZALEZ GRASSI, Palma de Mallorca (ES); Héloïse COUTELIER, Paris (FR); Victor Claude Léon SAYOUS, Paris (FR)

(21) Appl. No.: 18/575,552

(22) PCT Filed: Jul. 9, 2022
(86) PCT No.: PCT/EP2022/069207

§ 371 Date: Dec. 29, 2023

Related U.S. Application Data
(60) Provisional application No. 63/220,158, filed on Jul. 9, 2021.

(30) Foreign Application Priority Data
Jul. 9, 2021 (EP) 21305964.5

(51) Int. Cl. C12N 5/077; A23L 13/00

(52) U.S. Cl. CPC C12N 5/0658; A23L 13/00; C12N 2506/45; C12N 2510/00

(57) ABSTRACT

The invention belongs to the field of food biotechnology. More precisely the invention relates to so-called lab grown meat. The invention relates to a method for producing foodstuff comprising a step of processing in vitro differentiated non-human animal cells wherein said in vitro differentiated non-human animal cells originate from at least one oligopotent stem cell (OSC), said at least one OSC being inactivated for the expression of at least one lineage specifier gene. The invention also relates to said foodstuff and OSCs useful for producing said foodstuff.

### FIELD OF THE INVENTION

The invention relates to the fields of food production and more particularly to improved methods for producing foodstuff, based on in vitro grown non-human animal cells. Invention relates also to specific Oligopotent Stem Cells (OSCs) stably inactivated for at least one lineage specifier gene or combinations thereof.

### BACKGROUND OF THE INVENTION

The global population is expected to reach 9.7 billion in 2050 (United Nations, 2019), mainly due to growth of the population of mid and low-income countries. This growth together with changes in consumer's lifestyle and the improvement of living standard put a global stress on livestock management at the level of the planet. An estimate of more than a doubling in meat product consumption is expected between 2010 and 2050 in developing countries and more than 73% for the entire world (FAO, 2011). To face the environmental (in terms of pollution and management of natural resources) and geostrategic impacts of such increase there is an urgent need for an alternative, environmentally sustainable, meat production mode. Intensive animal farming does not fulfil these goals and also raises the issues of both food quality and animal welfare, which is an increasing concern in many societies. Meat produced in vitro offers a realistic alternative to meat from slaughtered animals for those who wish to consume it sustainably and/or have concerns about animal welfare.

One strategy for producing so-called in vitro meat or lab-grown meat, is to produce muscle fibers by culturing muscle stem cells, which are obtained from tissue samples from animals. Due to limited in vitro proliferation potential and lineage restriction of muscle stem cells, this strategy is unlikely to reach suitable yields of production, and would only allow obtaining food products derived from muscle. Further, a mere mass of skeletal muscle cells is far from reproducing the cellular content, architecture and texture of natural meat, which is composed by a variety of cell types including adipocytes, muscles, nerves, endothelial cells, just to cite a few.

Another strategy is to start from pluripotent stem cells (PSCs). These cells hold two fundamental properties, beneficial for foodstuff production. The first is self-renewal capacity, which allows expanding them indefinitely in vitro and therefore scaling up their production. The second is pluripotency, referring to their ability to differentiate into any adult cell type (skin, lungs, pancreas, liver, gastro-intestinal and uro-genital tracts, kidneys, bones, muscles, heart, vessels). Use of PSCs therefore provides the possibility to obtain any cell type useful for engineering meat-based or processed meat food products mimicking to a large extent the cellular composition of their physiological counterpart. WO2020/104650 discloses a method for generating large amounts of avian embryonic stem cells with low to no serum and growth factors requirements and the use of these cells in foodstuff production, but without including any differentiation step. In the absence of specific signalling cues, 3D culture of these cells results in the formation of so-called embryoid bodies which are cell aggregates made of an heterogeneous mass of differentiated cells displaying cell derivatives of the three embryonic germ layers, without means to control their respective quantity or quality (i.e. differentiation level and cellular diversity). Therefore, this approach is not amenable for the mass production of a specific differentiated cell type or of a mix thereof. WO2021/048325 discloses the use of avian stem cells as an ingredient of a food product and does not describe using cells obtained through in vitro differentiation of stem cells or oligopotent stem cells.

Lineage commitment is orchestrated at the genetic level by lineage-specific sets of transcription factors (lineage specifiers), responsible for activating and/or repressing directly or indirectly large sets of downstream target genes, implementing specific developmental programs towards a specific cell type of a particular organ. In some instances, culture conditions (support, shear stress, . . . ) can contribute to the differentiation processes. Starting from pluripotent cells, two main strategies are considered in the art to drive PSC differentiation into specific differentiated cell types. The first one, known as “directed differentiation”, consists in a multistep culturing protocol, comprising submitting the pluripotent cells to specific external signalling cues such as molecules added or removed from the culture media at defined time points depending on the final cell type desired, and this according to the succession of signals to which cells are submitted in the natural differentiation process. Advantageously, this does not require genetic modification of the cells, but the main drawback of this strategy is a somewhat cumbersome process and the use of costly recombinant growth factors, cytokines, hormones, or other small molecule compounds used for activating or inhibiting specific developmental pathways at different time points. The second one (namely “forced differentiation”) consists in genetically modifying pluripotent cells in order for these cells to express or overexpress the desired transcription factor(s) constitutively or upon a specific induction signal. Nonetheless, this method implies the insertion within the genome of the cells of foreign genetic material (a transgene) which needs to be stably expressed or remain inducible over cell generations in order to maintain the desired differentiation properties over generations of cells. Further to the problem of the stability and genetic rearrangements, induction of the transgenes or their maintenance within the genome of the cells might imply the exposure of the cell to antibiotics and/or chemicals or any other selection or induction means that are not desirable when considering producing food, particularly for human consumption. These strategies and current challenges of cultured meat were recently reviewed by Post et al. (2020). WO2015/066377 is interested in producing skeletal muscle cells from cell lines derived from livestock, which are modified to express an inducible myogenic transcription factor transgene. In some instances, cells are also genetically modified with an additional transgene allowing induced expression of pluripotency genes for mass production of cells before switching to the induction of differentiation into skeletal muscle cells. WO2018/227016 is interested in systems and methods aiming at producing cell cultured food products. As WO2015/066377, WO2018/227016 also describes genetically engineered cells for the transient and sequential expression of pluripotency and differentiation factor transgenes. Nevertheless, besides the above-mentioned drawbacks related to genetic stability of these cells, foodstuff with transgenic material has further to face consumer reluctance toward genetically engineered material, especially when comprising foreign genetic material.

Facing the challenge of feeding the future world, and the growing concern of animal welfare, in vitro-(cultured) meat remains a valuable solution. Nonetheless, there is still a need for cost effective (e.g., using less recombinant growth factors, cytokines hormones, small chemical compounds, or antibiotics) methods to produce meat foodstuff, based on in vitro differentiated non-human animal cell type, that approaches structural and organoleptic properties of conventional meat products.

### SUMMARY

Inventors have been able to set up non-human oligopotent stem cells (OSCs) that are useful for producing lab grown meat. OSCs are self-renewing stem cells which retain a capacity to differentiate into a limited number of cell lineages. These cells can be grown at yields that are compatible with foodstuff production. Cells are differentiated with no or less costly recombinant molecules than methods of the art, which is of a particular advantage. They are then transformed or incorporated into a foodstuff, these OSCs are inactivated for the expression of at least one lineage specifier gene.

Accordingly, the invention relates to a method for producing foodstuff which comprises a step of processing in vitro differentiated non-human animal cells wherein said in vitro differentiated non-human animal cells originate from at least one OSC, said at least one OSC being inactivated for the expression of at least one lineage specifier gene.

Said method can further comprise, prior to the step of processing in vitro differentiated non-human animal cells, a step of producing said in vitro differentiated non-human animal cells which comprises:
a step of amplifying at least one OSC inactivated for the expression of at least one lineage specifier gene,
optionally a step of culturing said amplified OSC as embryoid bodies, or
optionally, a step of differentiating said OSC.

This allows to obtain cells of interest at a rate sufficient for foodstuff production, thereby further lowering cost of said lab-grown based foodstuff. In a particular embodiment of said method, prior to the step of amplifying the at least one OSC inactivated for the expression of at least one lineage specifier gene, a step of obtaining said at least one OSC by stably inactivating at least one lineage specifier gene in a PSC or multipotent or totipotent cell by generating at least one insertion and/or deletion with a gene editing system. This allows generating cells with no exogenous genetic material inserted and thereby provides a safer and more stable approach compared to conventional genetically modified organisms (GMO) which incorporate foreign genetic material, sometimes seen as a threat, for instance by consumers.

Advantageously said OSC can be generated from a wide variety of PSCs that are available from many organisms. Accordingly, in a particular embodiment of the method, said PSC is selected from induced PSCs (iPSCs), embryonic stem cells (ESCs), nuclear transfer ESCs (ntESCs) from non-human animal origin. They can also be generated from multipotent or totipotent cells from non-human animal origin. Also, said PSC or multipotent or totipotent cells can originate from a non-human vertebrate, for example, a livestock, a fish, a bird; an insect; a crustacean, for example a shrimp, prawn, crab, crayfish, and/or a lobster; a mollusc, for example an octopus, squid, cuttlefish, scallops, snail, thereby paving the way for the production of a large variety of lab-grown foodstuffs.

Inventors surprisingly discover that instead of forcing cells to engage into a differentiation pathway through overexpression of one or several lineage specifier gene(s), forced differentiation can also be controlled through inactivation of particular lineage specifier genes promoting by defect other differentiation pathways. Therefore, in a particular embodiment of the method the at least one OSC is inactivated for the expression of at least one neurectoderm (NE), mesendoderm (MED), mesoderm (MD), or endoderm (ED) lineage specifier gene or a combination thereof. In a particular embodiment, said at least one NE, MED, MD, or ED lineage specifier gene is selected from PAX6, SOX1, ZNF521, SOX2, SOX3, ZIC1, TBXT, TBX6, MSGN1, KLF6, FOXA1, FOXA2, FOXA3, SOX17, HNF4A, GSC, MIXL1 and EOMES or a combination thereof. Further specific combinations of gene inactivations result in OSCs even further restricted into their differentiation capacities, which allow the cost-effective production of specific cell lineages. Also, in a more particular embodiment the OSC is inactivated for the expression of at least one NE lineage specifier gene and for the expression of at least one MD lineage specifier gene, thereby providing an ED restricted OSC. In another particular embodiment of the method said OSC is hepato-specific and is inactivated for the expression of at least one NE lineage specifier gene, for the expression of at least one MD lineage specifier gene and for the expression of at least one gene that governs differentiation of ED cells towards non-hepatic progenitor cells. In another particular embodiment of the method, said OSC is inactivated for the expression of at least one NE lineage specifier gene and for the expression of at least one ED lineage specifier gene. In a further particular embodiment of the method, said OSC is skeletal muscle specific and is inactivated for the expression of at least one NE lineage specifier gene, for the expression of at least one ED lineage specifier gene and for the expression of at least one gene that governs differentiation of MD cells towards non-skeletal muscle progenitor cell. In another particular embodiment of the method, said OSC is cardiac-specific and is inactivated for the expression of at least one NE lineage specifier gene, for the expression of at least one ED lineage specifier gene and for the expression of at least one gene that governs differentiation of MD cells towards non-cardiac progenitor cells. In another particular embodiment of the method, said OSC is adipocyte specific and is inactivated for the expression of at least one a NE lineage specifier gene, for the expression of at least one ED lineage specifier gene and for at least one gene that governs differentiation of MD cells towards non-adipocyte progenitor cells. In another particular embodiment of the method, said OSC is skin (keratinocyte) specific and is inactivated for the expression of at least one MED lineage specifier gene (or for at least one MD lineage specifier and at least one ED lineage specifier), and/or for the expression of at least one at least one gene that governs differentiation of NE cells towards non-skin progenitor cells.

Further, in the method for producing foodstuff, the at least one OSC is a skeletal muscle, cardiac, hepatocyte, fibroblast, red blood, keratinocyte, or adipocyte specific OSC, or a combination thereof, which allows to reproduce complex foodstuffs that mimics composition and organoleptic properties of meat (derived) products from farm animals.

Another object of the invention relates to a non-human OSC inactivated for the expression of at least two lineage specifier genes selected from the groups of NE, MD, ED or MED lineage specifiers genes.

A further object of the invention relates to a foodstuff comprising at least one non-human animal cell wherein the expression of at least one lineage specifier gene, selected from the groups of NE, MED, MD, or ED lineage specifiers genes, has been inactivated by generating at least one insertion and/or deletion with a gene editing system. This foodstuff provides a valuable and sustainable solution to the growing concern of feeding the world, while no presenting the drawbacks (e.g. high cost, low yields, or for GMOs: genetic instability, risk of dissemination of foreign DNA) of current lab grown meat.

### FIGURE LEGENDS

FIG. 1: PSCs differentiation pathways in vertebrates towards neurectoderm (NE), mesendoderm (MED), mesoderm (MD) and endoderm (ED) lineage or cell types. 1: set of genes governing differentiation towards NE lineage; 2: set of genes governing differentiation towards MED lineage; 3: set of genes governing differentiation from MED lineage cells towards ED lineage cells; 4 set of genes governing differentiation from MED lineage cells towards MD lineage cells; 5, 6, 7 sets of genes governing differentiation towards late NE, ED and MD lineage cells respectively.

FIG. 2: Gene Editing strategies to obtain early lineages (NE, MD, ED) restricted Oligopotent stem cells (OSCs. OSCs are self-renewing stem cells for which some differentiation pathways are barred by specific inactivation of lineage specifier gene, or combination thereof. A) example of a gene editing strategy according to the invention for obtaining a MD restricted OSC: at least one NE and at least one ED lineage specifier gene are inactivated. B) example of a gene editing strategy according to the invention for obtaining a NE restricted OSC: at least one ED I and at least one MD lineage specifier gene are inactivated. C) example of an alternative gene editing strategy according to the invention for obtaining a NE restricted OSC: at least one MED lineage specifier gene is inactivated. D) example of a gene editing strategy according to the invention for obtaining a ED restricted OSC: at least one MD and at least one NE lineage specifier gene are inactivated. Order of the generation of lineage specifier knock-out (ko) is only indicative. Do-not-enter signs in FIGS. 1 and 3) indicate differentiation pathways that are barred through lineage specifier ko in OSCs. EDMD OSC: An OSC, which differentiation potential has been restricted to ED, MD lineages, and their derivatives. NEMD OSC: An OSC, which differentiation potential has been restricted to NE, MD lineages, and their derivatives. NEED OSC: An OSC, which differentiation potential has been restricted to NE, ED lineages, and their derivatives. NE OSC: An OSC, which differentiation potential has been restricted to NE lineages and their derivatives. MD OSC: An OSC, which differentiation potential has been restricted to MD lineages and their derivatives. ED OSC: An OSC, which differentiation potential has been restricted to ED lineages and their derivatives.

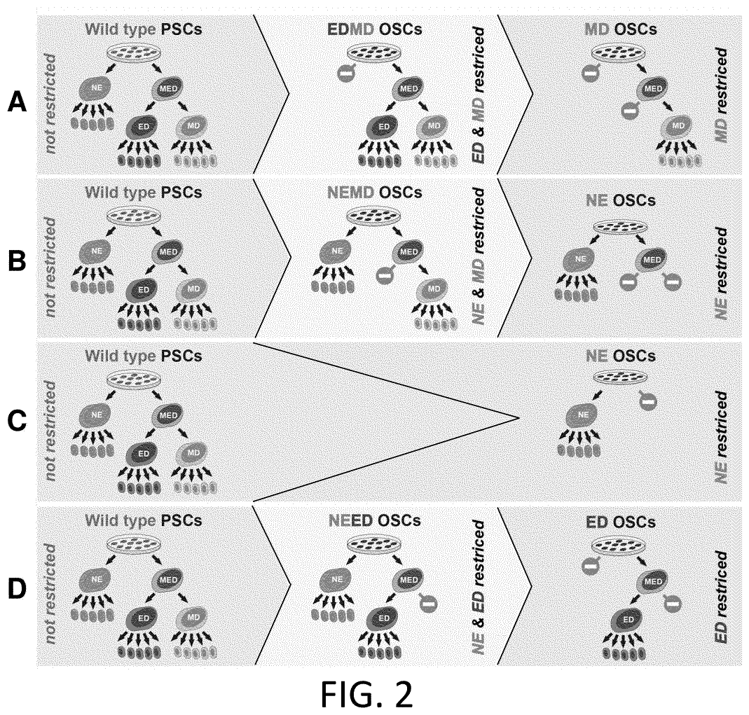

FIG. 3: Examples of gene editing strategies to obtain late lineages restricted OSCs, for example A) skin OSCs: several NE non-skin gene specifiers are inactivated by knock-out (ko) in a NE OSC, B) liver OSC: several ED non-hepatic tissue gene specifiers are inactivated by ko in a ED OSC, or C) heart OSCs: several MD non-heart tissue gene specifiers are inactivated by ko in a MD OSC. Do-not-enter signs indicate differentiation pathways that are barred or hindered in the OSC. Order of the generation of ko is only indicative.

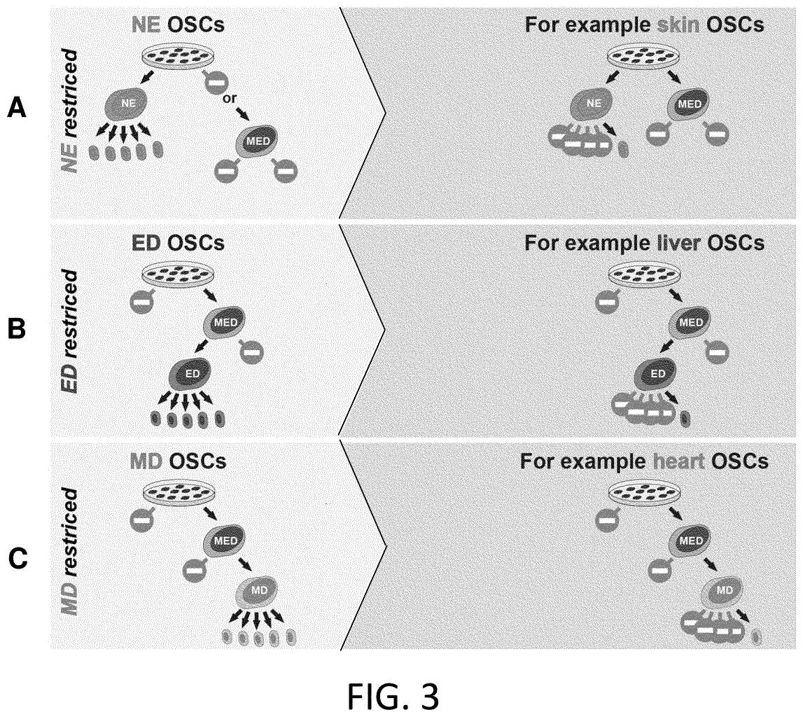

FIG. 4: % of CRISPR-edited Insertions and deletion (indels) in orthologous lineages specifier genes detected in the human iPSC-(A) and duck ESC-(B) pools used for generating edited OSC clonal lines. Single guide RNA (sgRNA).

FIG. 5: Multilineage embryoid body (EB) differentiation of wild type human iPSCs (hiPSCs). A) Image of an EB obtained from mammalian WTC-11 iPSCs. White bar: 200 μm. B) EB size at day 7 (D7) of EB differentiation. C) Pie chart lineage distribution obtained through quantification of pluripotent (PL), NE, ED, MD and MED lineage-specific transcripts at day 0 (DO) and D7 of differentiation obtained by reverse transcription followed by real time polymerase chain reaction (qRT-PCR).

FIG. 6: Day 14 EB differentiation potential of wild-type hiPSCs compared to edited NEMD, NE, EDMD and NEED human OSCs (hOSCs) demonstrating lineage differentiation biases resulting from genetic inactivation of single lineage specifier genes.

FIG. 7: Day 11 keratinocyte directed differentiation potential of wild-type hiPSCs compared to NE and EDMD hOSCs demonstrating keratinocyte differentiation biases resulting from genetic inactivation of single lineage specifier genes. Columns represent expression fold changes compared to wild type control gene expression at day 0 obtained through quantification of transcripts of two PL genes (NANOG, OCT4), one NE gene (PAX6), and two keratinocyte (K) genes (TP63, KRT14) at day 11 of keratinocyte differentiation obtained by qRT-PCR.

FIG. 8: Day 12 EB differentiation potential of wild-type dESCs compared to edited NEMD, EDMD and NEED duck OSCs (dOSCs) demonstrating lineage differentiation biases resulting from genetic inactivation of single orthologous lineage specifier genes. Pie chart lineage distribution obtained through quantification of duck PL, NE, ED, and MD lineage-specific transcripts at day 12 of differentiation obtained by qRT-PCR.

FIG. 9: Day 16 keratinocyte directed differentiation potential of wild-type dESCs compared to keratinocyte dOSCs demonstrating a skin differentiation bias resulting from simultaneous genetic inactivation of two orthologous lineage specifier genes: Pax6 (NE) and Gsc (MED). Columns represent expression fold changes compared to wild type control gene expression at day 0 obtained through qRT-PCR quantification of two PL genes (Nanog, Oct4), two NE genes (En1, Otx2), and two K genes (Tp63, Krt14).

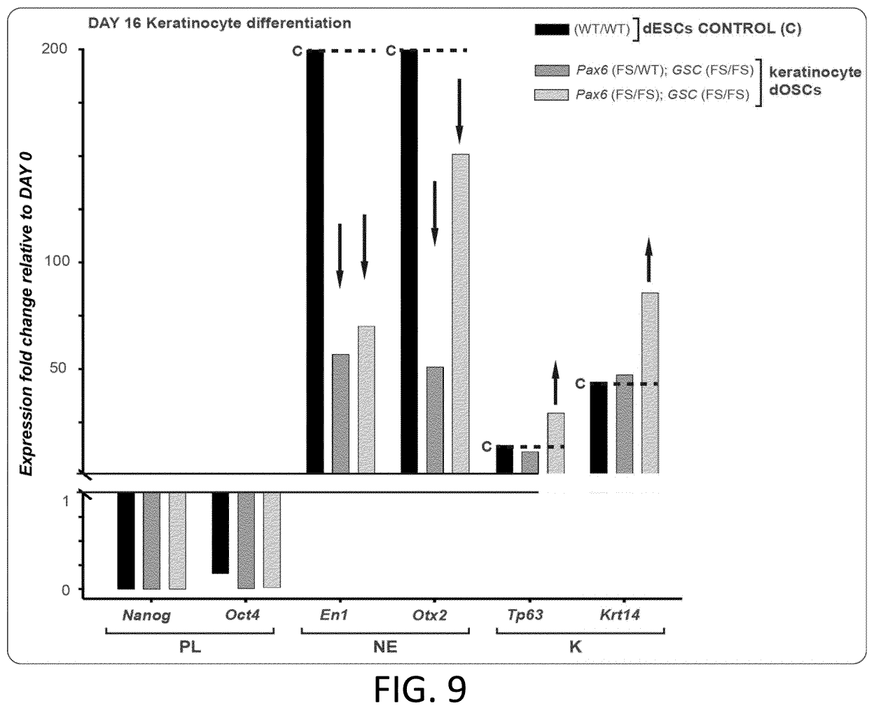

FIG. 10: Example of an embodiment of a method of producing a foodstuff using OSCs.

### DETAILED DESCRIPTION AND ADDITIONAL EMBODIMENTS

Invention relates, inter alia, to methods for producing foodstuff comprising in vitro differentiated non-human animal cells that originate from OSCs and are inactivated for the expression of at least one lineage specifier gene. Indeed, inventors have found that introducing a differentiation bias towards foodstuff-relevant cell types by restricting the potency of PSCs, multipotent or totipotent cells at early and/or late steps of differentiation results in OSCs that differentiate more efficiently, more homogeneously and require less exogenous factors than unmodified PSCs. This is obtained by stably inactivating early and/or late relevant lineage specifiers genes in said PSCs. Further, said OSCs do not contain exogenous genetic material and constitute more stable cell lines than transgenic PSC lines engineered for the forced ectopic expression of lineage specifier genes.

### Definitions

As used herein “Pluripotent Stem Cells” (PSCs) relate to cells that have the capacity to self-renew by dividing and to develop into the three primary germ cell layers (namely, the ED, the NE, and the MD) and their derivatives. In vertebrates, almost all cell types constituting the animal at birth originate from a subset of transiently pluripotent cells called the inner cell mass (ICM) in the mammalian blastocyst-stage pre-implantation embryo or the blastoderm (BDM) in oviparous vertebrates (birds, reptiles, amphibians, fishes). Mammalian ICM cell isolation and culture allows generating embryonic stem cell (ESC) lines showing self-renewal and pluripotency potential (Evans and Kaufman, 1981, Thomson et al., 1998). Similarly, BDM cells can also be easily isolated from early oviparous vertebrate embryos, to create pluripotent ESC lines. An alternative source of PSCs are animal somatic tissues through the process of reprogramming, either by ectopically expressing pluripotency-associated transcription factors in somatic cells (generating so-called induced PSCs or iPSCs see e.g., Takahashi and Yamanaka, 2006), or by generating ntESCs from cloned embryos obtained through injection of a somatic nucleus into an enucleated oocyte (Campbell et al., 1996). In recent years, ESC, iPSC and ntESC lines have been successfully derived from a wide spectrum of vertebrate species which are relevant for foodstuff production. Also, as used herein, the terms Pluripotent Stem Cells (PSC) designates any ESC, ntESC, iPSC, or blastomere isolated from the ICM, from any vertebrate relevant for food production, or combination thereof which retain the capacity to form cell-derivatives of the three embryonic germ layers. Accordingly, PSCs suitable for the invention are non-human animal cells.

Suitable PSCs encompass PSCs originating from any animal species that is commonly consumed in human or animal alimentation. In some instances, anyway, because the invention allows avoiding sacrificing said animals, it can also comprise any animal species that otherwise would be disregarded as a source of meat for human or animal alimentation, because of, for example economical concern, cultural habits or species scarcity. Also, said species comprise any non-human vertebrate, insect, crustacean or mollusc, in particular, those which are commonly consumed in human or animal alimentation. ‘Mollusc’ designates more particularly, but is not restricted to, octopus, squid, cuttlefish, scallops or a snail. ‘Insect’ designates more particularly, but is not restricted to, beetles (or other insects from the order of Coleoptera), butterflies or moths (or other insects from the order of Lepidoptera), bees, wasps or ants (or other insects from the order of Hymenoptera), grasshoppers, locusts or crickets (or other insects from the order of Orthoptera), cicadas, leafhoppers or planthoppers (or other insects from the order of Hemiptera). ‘Crustacean’ designates more particularly, but is not restricted to, shrimp, prawn, crab, crayfish, or a lobster. ‘Non-human vertebrates’ comprise any non-human mammals, fishes, amphibians, reptiles or birds, more particularly those commonly consumed in human or animal alimentation. Also, particularly preferred birds are those which are consumed for their meat or eggs; even more particularly said birds are a poultry selected from, but not restricted to: chicken, turkey, duck, goose, guinea fowl, pigeon, quail, squab or even pheasant, emu, swan, ostrich, parrots, finches, hawks, crows, and cassowary. Particularly preferred mammals are livestock bred for its meat, for example, bison, deer, kangaroo, horse, donkey, cattle, zebu, yak, buffalo, sheep, goat, reindeer, pig, wild boar, rabbit, guinea pig, llama. In a very particular embodiment, PSCs originate from any of the vertebrates selected from rabbit, guinea pig, cow, Arabian camel, goat, horse, pig, chicken, duck, gilthead seabream, European seabass, Atlantic cod and turbot.

“Oligopotent stem cells” (OSCs) are defined as progenitor cells which are pushed to differentiate into a few cell types and retain the capacity to self-renew indefinitely like PSCs. OSCs used for producing foodstuff according to the invention derive from PSCs as defined above, restricted in their differentiation potential toward a specific embryonic germ layer, organ progenitor and/or specific tissues. Accordingly, a MD specific OSC is an OSC which differentiation potential is restricted to, or at least strongly biased towards, cells of MD lineage, which constitute the bigger part or even the majority of the cells obtained through differentiation of said MD specific OSC. In other words, said cell lost its capability of, or is less prone to, differentiate into cells from NE and/or ED lineage in comparison with a PSC. Also, an ED specific OSC is an OSC which differentiation potential is restricted to, or at least strongly biased towards, cells of ED lineage which constitute the bigger part or even the majority of cells obtained through differentiation of said ED specific OSC. In other words, said cell lost its capability of, or is less prone to, differentiate into cells from NE and/or MD lineage in comparison with a PSC. Also, a NE specific OSC is an OSC which differentiation potential is restricted to, or at least strongly biased towards, cells of NE lineage which constitute the bigger part or even the majority of the cells obtained through differentiation of said NE specific OSC. In other words, said OSC lost its capability of, or is less prone to, differentiate into, cell lineages selected from MD and/or ED lineage in comparison with a PSC. As mentioned above, OSC can be restricted to differentiate into early lineage cell types; an OSC can also be restricted in its differentiation potential in order to differentiate preferentially towards cells of specific organs: for example a liver specific OSC (or liver OSC) is an OSC which differentiation potential is restricted to liver cells or progenitors thereof; a skeletal muscle specific OSC (or skeletal muscle OSC) is an OSC which differentiation potential is restricted to skeletal muscle cells or progenitors thereof; a cardiac-specific OSC (or heart OSC) is an OSC which differentiation potential is restricted to cardiac cells or progenitors thereof; a skin specific OSC (or skin OSC) is an OSC which differentiation potential is restricted to keratinocytes or progenitors thereof; an adipocyte specific OSC (or fat OSC) is an OSC which differentiation potential is restricted to adipocyte cells or progenitors thereof. As a corollary, an OSC of the invention which is restricted to a single or a reduced number of lineages refers to an OSC differentiating, when submitted to any differentiation protocol (e.g. EB or directed differentiation), into a cell population that is at least significantly enriched in cells from said lineage in comparison to a non-modified stem cell submitted to the same differentiation protocol. In a preferred embodiment, said population is increased by at least 10%, 20%, 30%, 40%, or even at least 50% in cells of said lineage in comparison to a non-modified stem cell submitted to the same differentiation protocol. Said lineage can be determined by any method known in the art, e.g. by quantitation of the expression of marker genes for differentiation lineages, at the transcriptional (qRT-PCR or any RNA quantitation method) or translational level (any protein quantitation method or cell sorting method).

MD, ED and ectoderm are the three primary germ layers of the early embryo. MED refers to cells from tissue layer which differentiate into MD or ED cells. Cells of MD lineage include cardiac and skeletal muscle cells, smooth muscle cells, non-epithelial blood cells and kidney cells. Also, a MD specific OSC will be biased towards differentiation into cardiac and/or skeletal muscle cells, smooth muscle cells, non-epithelial blood cells and/or kidney cells. ED differentiates to form interior linings, digestive glands and epithelia (e.g., gastrointestinal and respiratory tracts, liver, pancreas etc). Also, an ED specific OSC will be biased towards e.g., epithelial cells of digestive or respiratory tracts, liver and so on. Ectoderm differentiates to form epithelial (epidermal) tissues (e.g., skin, linings of the mouth, anus, nostrils, sweat glands, hair and nails, and tooth enamel) and neural tissues (central nervous system and peripheral nerves). Ectoderm derives from NE, which is the first step in the development of the nervous system and epithelial/epidermal tissues as exposed above. Also, an epidermal specific OSC will be biased towards skin cells or dander cells, more preferably skin cells and a neuroectoderm specific OSC will be biased towards ectoderm lineage, that are nervous cells and epithelial/epidermal cells.

As used herein, the terms “lineage specifier gene” refer to a gene encoding a transcription factor that is involved in the direct or indirect activation or repression of sets of several downstream target genes implementing specific developmental programs. Progression towards the different stages of embryonic development relies on the orchestrated activation of these developmental master genes triggered by tissue-tissue, cell-cell interactions and the activity of soluble signalling molecules (e.g., growth factors) secreted by the surrounding tissues/cells. Noteworthy, a high level of conservation of the genetic pathways and morphogenetic mechanisms governing embryonic development is observed in vertebrates from fishes to humans; indeed the general organization of their body plan, organs, cell types are highly similar. Also, it should be noticed that species from vertebrates, molluscs, insects and crustaceans, all are triploblastic, which means they all contain ectoderm, MD and ED, making generating OSCs feasible in those species. Therefore, orthologs for lineage specifier genes are present in foodstuff relevant species and can be easily retrieved in genome databases as e.g., Ensembl (website: ensembl.org/index.html). Also, although lineage specifier genes as listed in tables 1-4 or in this whole document are designated according to their name in H. sapiens, they are thought to designate any ortholog gene in any foodstuff relevant species. In a particular embodiment an ortholog for a “lineage specifier gene” encodes a protein whose sequence shares at least 30% homology in its amino acid sequence with the corresponding protein in H. sapiens, preferably, more than 30%, preferably 31%, 32%, 33%, 34%, 35%, 36%, 37%, 38%, or even 39%, preferably said sequence share is at least of 40%, preferably more than 40%, preferably 41%, more preferably 42%, 43%, 44%, 45%, 46%, 47%, 48%, even more preferably 49%, preferably said sequence share is at least of 50%, preferably more than 50%, preferably 51%, 52%, 53%, 54%, 55%, 56%, 57%, 58%, even more preferably 59%, preferably said sequence share is at least 60%, preferably more than 60%, preferably 61%, 62%, more preferably 63%, 64%, 65%, 66%, 67%, 68%, even more preferably 69%, preferably said sequence share is at least 70%, preferably more than 70%, preferably 71%, 72%, 73%, 74%, 75%, 76%, 77%, 78%, even more preferably 79%, preferably, said sequence share is at least 80%, preferably more than 80%, preferably 81%, 82%, 83%, 84%, 85%, 86%, 87%, 88%, even more preferably 89%, preferably said sequence share is at least 90%, preferably more than 90%, preferably 91%, 92%, 93%, 94%, 95%, 96% 97%, 98% and even more preferably more than 99% of homology. In another particular embodiment an ortholog for a “lineage specifier gene” encodes a protein whose sequence share is at least 50%, preferably more than 50%, preferably 51%, 52%, 53%, 54%, 55%, 56%, 57%, 58%, even more preferably 59%, preferably said sequence share is at least 60%, preferably more than 60%, preferably 61%, 62%, more preferably 63%, 64%, 65%, 66%, 67%, 68%, even more preferably 69%, preferably said sequence share is at least 70%, at least 70% homology in their amino acid sequence with the corresponding functional domain of the protein in H. sapiens, preferably, more than 70%, preferably 71%, more preferably 72%, 73%, 74%, 75%, 76%, 77%, 77%, 79%, even more preferably, more than 80%, preferably 81%, more preferably 82%, 83%, 84%, 85%, 86%, 87%, 88%, 89%, 90%, 91%, 92%, 93%, 94%, 95%, 96%, 97%, 98%, 99% even more preferably 100% homology.

TABLE 1

| NE lineage specifier genes (human gene name) | | Ensembl ID |
|---|---|---|
| early NE | PAX6 | ENSG00000007372 |
| early NE | SOX1 | ENSG00000182968 |
| early NE | ZNF521 | ENSG00000198795 |
| early NE | SOX2 | ENSG00000181449 |
| early NE | SOX3 | ENSG00000134595 |
| early NE | ZIC1 | ENSG00000152977 |
| neural lineage | NEUROD2 | ENSG00000133937 |
| neural lineage | SOX10 | ENSG00000185155 |
| neural lineage | PAX3 | ENSG00000163508 |
| neural lineage | PAX6 | ENSG00000007372 |

TABLE 2
ED lineage specifier genes

| | human | |
|---|---|---|
| lineage/cell type | gene name | Ensembl ID |
| early ED | FOXA1 | ENSG00000129514 |
| early ED | FOXA2 | ENSG00000125798 |
| early ED | FOXA3 | ENSG00000170608 |
| early ED | SOX17 | ENSG00000164736 |
| early ED | HNF4A | ENSG00000101076 |
| early extraembryonic ED | SOX7 | ENSG00000171056 |
| cardiac-inducing ED | GATA4 | ENSG00000136574 |
| cardiac-inducing ED | GATA5 | ENSG00000130700 |
| cardiac-inducing ED | GATA6 | ENSG00000141448 |
| foregut: pyloris, duodenum, pancreas | PDX1 | ENSG00000139515 |
| anterior foregut, ED, lung, thyroid | NKX2.1 | ENSG00000136352 |
| anterior foregut, ED, thymus | FOXN1 | ENSG00000109101 |
| mid/hindgut - intestinal epithelia | CDX2 | ENSG00000165556 |

TABLE 3

| MED lineage specifier (human gene name) | Ensembl ID |
|---|---|
| GSC | ENSG00000133937 |
| MIXL1 | ENSG00000185155 |
| EOMES | ENSG00000163508 |

TABLE 4
mesoderm lineage specifier genes

| lineage/cell type | gene name | Ensembl ID |
|---|---|---|
| early MD | KLF6 | ENSG00000067082 |
| early MD | TBXT | ENSG00000164458 |
| early MD | TBX6 | ENSG00000149922 |
| early MD | MSGN1 | ENSG00000151379 |
| endothelial cells | SOX18 | ENSG00000203883 |
| kidney | OSR1 | ENSG00000143867 |
| kidney | EYA1 | ENSG00000104313 |
| hematopoietic lineage | RUNX1 | ENSG00000159216 |
| hematopoietic lineage | TAL1 | ENSG00000162367 |
| heart - cardiac MD | MESP1 | ENSG00000127241 |
| heart - primary and | NKX2.5 | ENSG00000183072 |
| secondary heart fields | | |
| heart - secondary heart field | ISL1 | ENSG00000016082 |
| skeletal muscle - Myogenesis | PAX7 | ENSG00000009709 |
| skeletal muscle - Myogenesis | MYOD | ENSG00000129152 |
| skeletal muscle - Myogenesis | MYF5 | ENSG00000111049 |
| bone - Osteogenesis | RUNX2 | ENSG00000124813 |
| bone - Osteogenesis | KLF2 | ENSG00000127528 |
| bone - Osteogenesis | MSX2 | ENSG00000120149 |
| cartilage - chondrogenesis | PAX9 | ENSG00000198807 |
| cartilage - chondrogenesis | NKX3.2 | ENSG00000109705 |
| cartilage - chondrogenesis | SOX8 | ENSG00000005513 |
| cartilage - chondrogenesis | SOX9 | ENSG00000125398 |
| fat - adipogenesis | PPARG (PPARγ) | ENSG00000132170 |
| fat - adipogenesis | CEBPA | ENSG00000245848 |

The terms “restricted to” or “biased to”, when related to an OSC, are used interchangeably, and mean that said OSC differentiate more efficiently, homogeneously, mainly and/or spontaneously to a specific lineage or a differentiation stage.

The terms “in vitro meat”, “lab-grown meat”, “synthetic meat” are used herein interchangeably and designate cells, cell mass, tissue, reconstituted or not, resulting from culturing, differentiating and/or processing OSCs described herein.

“Foodstuff” encompasses any fresh product, dried product, frozen product, powder, paste, extrudate, a liquid product or a solid product resulting from the processing of differentiated OSCs, adapted to be minced, dried, cooked, done, rehydrated, pickled or smoked. “Foodstuff” also encompasses food product defined, for example, as a soup, a sauce, a stew, a topping, a seasoning, a sausage, minced meat, a meatball, a nugget, a spread, a pâté, a puree, a drink or shake, a surimi, a bar, a biscuit, dried granules, tablets, capsules, a powder. In a particular embodiment foodstuff also comprises shakes, powders, bars to be used, e.g., as a food supplement.

### Oligopotent Stem Cells

Inventors have discovered that PSCs, multipotent or totipotent cells inactivated for the expression of at least one lineage specifier gene result in oligopotent stem cells (OSCs) that differentiate in vitro more efficiently, more homogeneously and/or require less exogenous factors than unmodified PSCs, multipotent or totipotent cells and thereby constitute advantageous tools for producing foodstuffs comprising in vitro differentiated non-human animal cells.

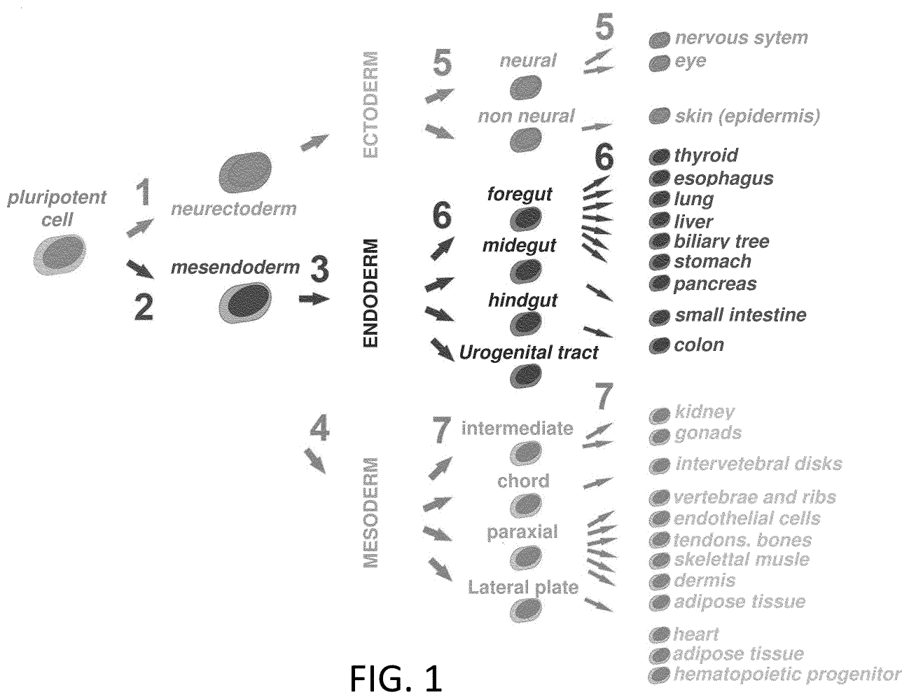

The differential activation of lineage specifier genes is responsible for activating/repressing directly or indirectly large sets of downstream target genes, which implement specific developmental programs towards the primary germ layers and then towards specific differentiated cell types. Lineage specifier genes constitute genetic switches in which transcription factor lineage specifier genes set 1 expression specifies NE, while lineage specifier genes set 2 specifies MED (i.e. MD and ED), and so on until organogenesis is completed (FIG. 1).

Accordingly, an object of the invention relates to an OSC that is inactivated for the expression of at least one early lineage specifier gene required for the specification and differentiation of cells of one of the early embryonic germ layers (ED, MD, MED and NE). More particularly, said object relates to an OSC that is inactivated for the expression of at least one gene selected from PAX6, SOX1, ZNF521, SOX2, SOX3, ZIC1, TBXT, TBX6, MSGN1, KLF6, FOXA1, FOXA2, FOXA3, SOX17, HNF4A, GSC, MIXL1 and EOMES or a combination thereof. Even particularly, said object relates to an OSC that is inactivated for the expression, of at least one gene selected from PAX6, SOX1, TBXT, TBX6, FOXA2, SOX17, GSC, MIXL1 and EOMES or a combination thereof. A more particular object of the invention is an OSC that is inactivated for the expression of at least two lineage specifier genes selected from the groups of ED, MD, MED or NE lineage specifiers genes. In that regard, said object relates to an OSC that is inactivated for the expression of at least two genes selected from PAX6, SOX1, ZNF521, SOX2, SOX3, ZIC1, TBXT, TBX6, MSGN1, KLF6, FOXA1, FOXA2, FOXA3, SOX17, HNF4A, GSC, MIXL1 and EOMES.

In an embodiment, said OSC is inactivated for the expression of at least one early NE lineage specifier gene. In a particular embodiment said at least one NE lineage specifier gene is selected from PAX6, SOX1, ZNF521, SOX2, SOX3 and ZIC1, or a combination thereof. The resulting OSCs are advantageously restricted to differentiate into cells from MD and/or ED lineage.

In another embodiment, said OSC is inactivated for the expression of at least one early MD lineage specifier gene. In a particular embodiment said at least one MD lineage specifier gene is selected from TBXT, TBX6, MSGN1 and KLF6, or a combination thereof. The resulting OSCs are advantageously restricted to differentiate into cells from NE and/or ED lineage.

In another embodiment, said OSC is inactivated for the expression of at least one early ED lineage specifier gene. In an even more particular embodiment, said at least one ED lineage specifier gene is selected from FOXA1, FOXA2, FOXA3, SOX17, and HNF4A, or a combination thereof. The resulting OSCs are advantageously restricted to differentiate into cells from NE and/or MD lineage.

In further embodiment, said OSC is inactivated for the expression of at least one MED lineage specifier gene. In an even more particular embodiment, said at least one MED lineage specifier gene is selected from GSC, MIXL1 or EOMES, or a combination thereof. The resulting OSCs are advantageously restricted to differentiate into cells of NE lineage.

### OSC Restricted or Biased Towards NE Lineage:

In a particular embodiment, said OSC is inactivated for the expression of at least one MED lineage specifier gene. In an even more particular embodiment, said at least one MED lineage specifier gene is selected from GSC, MIXL1 and EOMES, or a combination thereof. In a more particular embodiment, said OSC is inactivated for the expression of GSC, MIXL1 and EOMES. These OSCs inactivated for the expression of at least one MED lineage specifier gene are advantageously restricted to differentiate into cells from NE lineage. In a more particular embodiment, the OSC is inactivated for the expression of at least one MED lineage specifier gene and of at least one late neural lineage specifier gene. Said OSC is advantageously restricted to, or biased towards, the epidermal (skin) lineage. In a more particular embodiment, in said OSC restricted to the epidermal lineage, the at least one MED lineage specifier gene expression of which is inactivated is selected from GSC, MIXL1 and EOMES, or a combination thereof. In a very particular embodiment, said OSC restricted to the epidermal lineage is inactivated for the expression of GSC, MIXL1 and EOMES In another particular embodiment, in said OSC restricted to the epidermal (skin) lineage, the at least one neural lineage specifier gene expression of which is inactivated is selected from NEUROD2, SOX10, PAX3 and PAX6, or a combination thereof. Hence, in a particular embodiment, in said OSC restricted to the epidermal lineage OSC is inactivated for the expression of at least one gene selected from GSC, MIXL1 and EOMES and at least one gene selected from NEUROD2, SOX10, PAX3 and PAX6. In a very particular embodiment, said OSC restricted to the epidermal lineage is inactivated for the expression of NEUROD2, SOX10, PAX3 and PAX6. In a further particular embodiment, said OSC restricted to the epidermal (skin) lineage is inactivated for the expression of GSC, MIXL1, EOMES, NEUROD2, SOX10, PAX3 and PAX6.

In another particular embodiment, said OSC is inactivated for the expression of at least one early MD lineage specifier gene and of at least one early ED lineage specifier gene. Said OSC is then also restricted to, or biased towards, the NE lineage. It can be further inactivated for the expression of at least one late neural lineage, said OSC is thereby advantageously restricted to, or biased towards, the epidermal (skin) lineage. In a more particular embodiment, in said OSC restricted to the epidermal lineage, the at least one early MD lineage specifier gene which expression is inactivated is selected from TBXT, TBX6, MSGN1 and KLF6, or a combination thereof. In a further particular embodiment, said OSC restricted to the epidermal lineage is inactivated for the expression of TBXT, TBX6, MSGN1 and KLF6. In another particular embodiment, in said OSC restricted to the epidermal lineage, the at least one early ED lineage specifier gene which expression is inactivated is selected from FOXA1, FOXA2, FOXA3, SOX17, and HNF4A, or a combination thereof. In a further particular embodiment, said OSC restricted to the epidermal lineage is inactivated for the expression of FOXA1, FOXA2, FOXA3, SOX17, and HNF4A. In another particular embodiment, in said OSC restricted to the epidermal (skin) lineage, the at least one neural lineage specifier gene expression of which is inactivated is selected from NEUROD2, SOX10, PAX3 and PAX6, or a combination thereof. In a very particular embodiment, said OSC restricted to the epidermal lineage is inactivated for the expression of NEUROD2, SOX10 and PAX3. In a further particular embodiment, said OSC restricted to the epidermal lineage is inactivated for the expression of NEUROD2, SOX10, PAX6 and PAX3. In an even more particular embodiment, said OSC restricted to the epidermal (skin) lineage is inactivated for the expression of TBXT, TBX6, MSGN1, KLF6 FOXA1, FOXA2, FOXA3, SOX17, HNF4A, NEUROD2, SOX10 and PAX3. In another particular embodiment, said OSC restricted to the epidermal (skin) lineage is inactivated for the expression of TBXT, TBX6, MSGN1, KLF6 FOXA1, FOXA2, FOXA3, SOX17, HNF4A, NEUROD2, SOX10, PAX6 and PAX3.

In another particular embodiment, an OSC restricted to the epidermal (skin) lineage, can be an OSC in which at least one neural lineage specifier is inactivated. In a more particular embodiment said neural lineage specifier is selected from NEUROD2, SOX10, PAX6 and PAX3, or a combination thereof.

Indeed said OSCs restricted or biased towards NE lineage or epidermal lineage are naturally inclined to differentiate into NE or epidermal lineage cells and therefore require less (than in usual in vitro differentiation protocols) or no need for specific factors for promoting cell differentiation into cells of NE or epidermal lineage which is of a particular advantage while considering using these cells to produce foodstuff. In an embodiment said OSCs restricted or biased towards NE lineage or epidermal lineage require 2, 3, 4, 5, 6, 7, 8, 9, 10, 20, 30, 40, 50, 100, 1000 time less or even no exogenous factors to differentiate into cells of NE or epidermal lineage.

### OSC Restricted or Biased Towards ED Lineage:

In a particular embodiment, said OSC is inactivated for the expression of at least one early NE lineage specifier gene and for the expression of at least one MD lineage specifier gene. Said OSC is advantageously restricted to, or biased towards, the ED lineage. In a more particular embodiment, in said OSC restricted to the ED lineage, the at least one early NE lineage specifier gene expression of which is inactivated is selected from PAX6, SOX1, ZNF521, SOX2, SOX3 and ZIC1, or a combination thereof. In an even more particular embodiment, said OSC restricted to the ED lineage is inactivated for the expression of PAX6, SOX1, ZNF521, SOX2, SOX3 and ZIC1. In another particular embodiment, in said OSC restricted to the ED lineage, the at least one MD lineage specifier expression of which is inactivated is selected from TBXT, TBX6, MSGN1 and KLF6, or a combination thereof. In an even more particular embodiment, said OSC restricted to the ED lineage is inactivated for the expression of TBXT, TBX6, MSGN1 and KLF6. In a further particular embodiment, in said OSC restricted to the ED lineage is inactivated for the expression of at least one early NE lineage specifier gene selected from PAX6, SOX1, ZNF521, SOX2, SOX3 and ZIC1, or a combination thereof, and for the expression of at least one MD lineage specifier gene selected from TBXT, TBX6, MSGN1 and KLF6, or a combination thereof. In a very particular embodiment, said OSC restricted to the ED lineage is inactivated for the expression of PAX6, SOX1, ZNF521, SOX2, SOX3, ZIC1, TBXT, TBX6, MSGN1 and KLF6. A preferred OSC restricted to the ED lineage is inactivated for the expression of at least PAX6, and for the expression of at least one gene selected from TBXT, TBX6, MSGN1 and KLF6, or a combination thereof. Another preferred OSC restricted to the ED lineage is inactivated for the expression of at least SOX1 and for the expression of at least one gene selected from TBXT, TBX6, MSGN1 and KLF6, or a combination thereof. Another preferred OSC restricted to the ED lineage is inactivated for the expression of at least ZNF521 and for the expression of at least one gene selected from TBXT, TBX6, MSGN1 and KLF6, or a combination thereof. Another preferred OSC restricted to the ED lineage is inactivated for the expression of at least SOX2 and for the expression of at least one gene selected from TBXT, TBX6, MSGN1 and KLF6. Another preferred OSC restricted to the ED lineage is inactivated for the expression of at least SOX3 and for the expression of at least one gene selected from TBXT, TBX6, MSGN1 and KLF6, or a combination thereof. Another preferred OSC restricted to the ED lineage is inactivated for the expression of at least ZIC1 and for the expression of at least one gene selected from TBXT, TBX6, MSGN1 and KLF6, or a combination thereof.

Said OSC restricted to the ED lineage differentiates into ED cells with less (than in usual in vitro differentiation protocols) or no need for ED specific factors which is of a particular advantage while considering using these cells to produce foodstuff. In particular, said OSCs restricted to the ED lineage are biased to differentiate into specific differentiated cells such as early extraembryonic ED, cardiac-inducing ED, liver, pancreas, midgut and hindgut, pyloris, duodenum, pancreas, anterior foregut ED, lung, thyroid, thymus, mid/hindgut, intestinal epithelial cells, or in principle any other ED-derived lineage.

In a particular embodiment an OSC restricted to ED lineage as described above is further inactivated for the expression of at least one gene selected from: SOX7, GATA4, GATA5, GATA6, PDX1, NKX2.1, FOXN1 and CDX2, or a combination thereof.

In a very particular embodiment, an OSC restricted to ED lineage as described in any of paragraphs [0045-0046] is further inactivated for the expression of at least one gene that governs differentiation of ED cells towards non-hepatic lineage cells. In an even more particular embodiment said at least one gene selected from: SOX7, GATA4, GATA5, GATA6, PDX1, NKX2.1, FOXN1 and CDX2 or a combination thereof. In a further particular embodiment said OSC restricted to ED lineage is inactivated for the expression of SOX7 and of at least one gene selected from GATA4, GATA5 and GATA6, or a combination thereof thereby providing an OSC restricted to differentiation into hepatocyte. In a further particular embodiment said OSC restricted to ED lineage is inactivated for the expression of at least SOX7 and at least one gene selected from: PDX1, NKX2.1, FOXN1 and CDX2, or a combination thereof, thereby providing an OSC restricted to differentiation into hepatocyte. In a further particular embodiment, said OSC restricted to ED lineage is inactivated for the expression of SOX7, GATA4, GATA5, GATA6, FOXA3, PDX1, NKX2.1, FOXN1 and CDX2, thereby providing an OSC restricted to differentiation into hepatocyte.

In another particular embodiment, an OSC restricted to differentiation into a hepatocyte can be an OSC in which at least one gene that governs differentiation of ED cells towards non-hepatic lineage cells neural lineage specifier is inactivated. In a more particular embodiment said at least one gene is selected from SOX7, GATA4, GATA5, GATA6, PDX1, NKX2.1, FOXN1 and CDX2 or a combination thereof.

### OSC Restricted or Biased Towards MD Lineage:

In another particular embodiment, said OSC is inactivated for the expression of at least one early NE lineage specifier gene and for the expression of at least one ED lineage specifier gene. Said OSC is advantageously restricted to, or biased towards, the MD lineage. In a more particular embodiment, in said OSC restricted to the MD lineage, the least one early NE lineage specifier gene expression of which is inactivated is selected from PAX6, SOX1, ZNF521, SOX2, SOX3 and ZIC1, or a combination thereof. In an even more particular embodiment, said OSC restricted to the MD lineage is inactivated for the expression of PAX6, SOX1, ZNF521, SOX2, SOX3 and ZIC1. In another particular embodiment, in said OSC restricted to the MD lineage, the at least one ED lineage specifier gene expression of which is inactivated is selected from FOXA1, FOXA2, FOXA3, SOX17, and HNF4A, or a combination thereof. In an even more particular embodiment, said OSC restricted to the MD lineage is inactivated for the expression of FOXA1, FOXA2, FOXA3, SOX17, and HNF4A. In another particular embodiment, said OSC restricted to the MD lineage is inactivated for the expression of at least one early NE lineage specifier gene selected from PAX6, SOX1, ZNF521, SOX2, SOX3 and ZIC1, or a combination thereof and for the expression of at least one ED lineage specifier gene selected from FOXA1, FOXA2, FOXA3, SOX17, and HNF4A, or a combination thereof. A preferred OSC restricted to the MD lineage is inactivated for the expression of at least PAX6, and for the expression of at least one gene selected from FOXA1, FOXA2, FOXA3, SOX17 and HNF4A, or a combination thereof. Another preferred OSC restricted to the MD lineage is inactivated for the expression of at least SOX1, and for the expression of at least one gene selected from FOXA1, FOXA2, FOXA3, SOX17 and HNF4A, or a combination thereof. Another preferred OSC restricted to the MD lineage is inactivated for the expression of at least ZNF521, and for the expression of at least one gene selected from FOXA1, FOXA2, FOXA3, SOX17 and HNF4A, or a combination thereof. Another preferred OSC restricted to the MD lineage is inactivated for the expression of at least SOX2, and for the expression of at least one gene selected from FOXA1, FOXA2, FOXA3, SOX17 and HNF4A, or a combination thereof. Another preferred OSC restricted to the MD lineage is inactivated for the expression of at least SOX3, and for the expression of at least one gene selected from FOXA1, FOXA2, FOXA3, SOX17 and HNF4A, or a combination thereof. Another preferred OSC restricted to the MD lineage is inactivated for the expression of at least ZIC1, and for the expression of at least one gene selected from FOXA1, FOXA2, FOXA3, SOX17 and HNF4A, or a combination thereof.

Said OSC restricted to the MD lineage differentiates into MD cells with less (than in usual in vitro differentiation protocols) or no need for MD specific factors which is of a particular advantage while considering using these cells to produce foodstuff. In particular, said OSCs restricted to the MD lineage are biased to differentiate into specific differentiated cells such as cells of endothelial lineage, kidney lineage, hematopoietic lineage (as e.g., red blood cells), skeletal muscle lineage (as skeletal myocytes), cardiac lineage (as cardiomyocytes), bone lineage, cartilage lineage, fat lineage (as adipocytes), fibroblast lineage.

In a particular embodiment an OSC restricted to MD lineage as described above is further inactivated for the expression of at least one gene selected from: SOX18, OSR1, EYA1, RUNX1, TAL1, MESP1, NKX2.5, ISL1, PAX7, MYOD1, MYF5, RUNX2, KLF2, MSX2, PAX9, NKX3.2, SOX8, SOX9, PPARG, and CEBPA, or a combination thereof.

In another particular embodiment, said OSC restricted to MD lineage as described in paragraphs [0049-0050] is inactivated for the expression of at least one gene that governs differentiation of MD cells towards non-cardiac progenitor cells, thereby providing a cardiac specific OSC, restricted to, or biased towards, differentiation into cardiomyocytes. In an even more particular embodiment said cardiac specific OSC is inactivated for the expression of at least one gene selected from SOX18, OSR1, EYA1, RUNX1, TAL1, PAX7, MYOD1, MYF5, RUNX2, KLF2, MSX2, PAX9, NKX3.2, SOX8, SOX9, PPARG, and CEBPA, or a combination thereof, thereby providing an OSC restricted to, or biased towards, differentiation into cardiomyocytes. In a further particular embodiment said OSC restricted to MD lineage is a cardiac specific OSC which is inactivated for the expression of SOX18, OSR1, EYA1, RUNX1, TAL1, PAX7, MYOD1, MYF5, RUNX2, KLF2, MSX2, PAX9, NKX3.2, SOX8, SOX9, PPARG, and CEBPA, thereby providing an OSC restricted to, or biased towards, differentiation into cardiomyocytes.

In another particular embodiment, an OSC restricted to differentiation into a cardiomyocyte can be an OSC in which at least one gene selected from SOX18, OSR1, EYA1, RUNX1, TAL1, PAX7, MYOD1, MYF5, RUNX2, KLF2, MSX2, PAX9, NKX3.2, SOX8, SOX9, PPARG, and CEBPA is inactivated.

In another particular embodiment, said OSC restricted to MD lineage as described in paragraphs [0049-0050] is inactivated for the expression of at least one gene that governs differentiation of MD cells towards non-skeletal muscle progenitor cells, thereby providing a skeletal muscle specific OSC, restricted to, or biased towards, differentiation into skeletal myocytes or a progenitor thereof. In a more particular embodiment said OSC restricted to MD lineage is inactivated for the expression of at least one gene selected from SOX18, OSR1, EYA1, RUNX1, TAL1, MESP1, NKX2.5, ISL1, RUNX2, KLF2, MSX2, PAX9, NKX3.2, SOX8, SOX9, PPARG, and CEBPA, or a combination thereof, thereby providing an OSC restricted to, or biased towards, differentiation into skeletal myocyte or a progenitor thereof. In an even more particular embodiment, said OSC restricted to MD lineage is inactivated for the expression of SOX18, OSR1, EYA1, RUNX1, TAL1, MESP1, NKX2.5, ISL1, RUNX2, KLF2, MSX2, PAX9, NKX3.2, SOX8, SOX9, PPARG, and CEBPA, thereby providing an OSC restricted to, or biased towards, differentiation into skeletal myocyte or a progenitor thereof.

In another particular embodiment, an OSC restricted to differentiation into a skeletal myocyte can be an OSC in which at least one gene selected from SOX18, OSR1, EYA1, RUNX1, TAL1, MESP1, NKX2.5, ISL1, RUNX2, KLF2, MSX2, PAX9, NKX3.2, SOX8, SOX9, PPARG, and CEBPA is inactivated.

In another particular embodiment, said OSC restricted to MD lineage as described in paragraphs [0049-0050] is inactivated for the expression of at least one gene that governs differentiation of MD cells towards non-adipocyte progenitor cells, thereby providing an adipocyte specific OSC, restricted to, or biased towards, differentiation into adipocyte. In a more particular embodiment said OSC restricted to, or biased towards, MD lineage is inactivated for the expression of at least one gene selected from SOX18, OSR1, EYA1, RUNX1, TAL1, MESP1, NKX2.5, ISL1, PAX7, MYOD1, MYF5, RUNX2, KLF2, MSX2, PAX9, NKX3.2, SOX8, and SOX9, or a combination thereof, thereby providing an OSC restricted to, or biased towards, differentiation into adipocyte. In an even more particular embodiment said OSC restricted to ED lineage is inactivated for the expression of SOX18, OSR1, EYA1, RUNX1, TAL1, MESP1, NKX2.5, ISL1, PAX7, MYOD1, MYF5, RUNX2, KLF2, MSX2, PAX9, NKX3.2, SOX8, and SOX9, thereby providing an OSC restricted to, or biased towards, differentiation into adipocyte.

In another particular embodiment, an OSC restricted to differentiation into an adipocyte can be an OSC in which at least one gene selected from SOX18, OSR1, EYA1, RUNX1, TAL1, MESP1, NKX2.5, ISL1, PAX7, MYOD1, MYF5, RUNX2, KLF2, MSX2, PAX9, NKX3.2, SOX8, and SOX9 is inactivated.

In another particular embodiment, said OSC restricted to MD lineage as described in paragraphs [0049-0050] is inactivated for the expression of at least one gene that governs differentiation of MD cells towards non-hematopoietic progenitor cells, thereby providing an hematopoietic specific OSC, restricted to, or biased towards, differentiation into hematopoietic cells. In a more particular embodiment said OSC restricted to MD lineage is inactivated for the expression of at least one gene selected from SOX18, OSR1, EYA1, MESP1, NKX2.5, ISL1, PAX7, MYOD1, MYF5, RUNX2, KLF2, MSX2, PAX9, NKX3.2, SOX8, SOX9, PPARG, and CEBPA, or a combination thereof, thereby providing an OSC restricted to, or biased towards, differentiation into hematopoietic cells. In an even more particular embodiment said OSC restricted to MD lineage is inactivated for the expression of SOX18, OSR1, EYA1, MESP1, NKX2.5, ISL1, PAX7, MYOD1, MYF5, RUNX2, KLF2, MSX2, PAX9, NKX3.2, SOX8, SOX9, PPARG, and CEBPA, thereby providing an OSC restricted to differentiation into hematopoietic cells.

In another particular embodiment, an OSC restricted to differentiation into a hematopoietic cell can be an OSC in which at least one gene selected from SOX18, OSR1, EYA1, MESP1, NKX2.5, ISL1, PAX7, MYOD1, MYF5, RUNX2, KLF2, MSX2, PAX9, NKX3.2, SOX8, SOX9, PPARG, and CEBPA is inactivated.

In another particular embodiment, an OSC according to the invention is inactivated for the expression of at least one gene, preferably two genes, selected from SOX18, OSR1, EYA1, RUNX1, TAL1, MESP1, NKX2.5, ISL1, PAX7, MYOD, MYF5, RUNX2, KLF2, MSX2, PAX9, NKX3.2, SOX8, SOX9, PPARG (PPARγ), CEBPA, GATA4, GATA5, GATA6, PDX1, NKX2.1, FOXN1, CDX2, NEUROD2, SOX10, PAX3 and PAX6.

### Methods of Producing a Foodstuff

Stem cells are considered valuable tools in the fields of transplantation therapy, regenerative medicine, and tissue engineering. Their use for producing meat in vitro is considered as an alternative to meat originating from animals in terms of sustainability but also of animal welfare. Anyway, the technology is still in its infancy and there is a need to use stable cells that are capable to bulk up sufficiently and then to differentiate in the desired cell type or progenitor thereof at a satisfying yield, while using as few as possible costly recombinant differentiation factors and numerous growth media. Inventors discovered that OSCs as described above are particularly suitable for in vitro meat production and foodstuff derived therefrom.

Accordingly, one object of the invention relates to a method for producing foodstuff comprising a step of processing in vitro differentiated non-human animal cells originating from at least one OSC inactivated for the expression of at least one lineage specifier gene.

Indeed, most of the strategies developed in the art consist in integrating inducible transgenes expressing lineage specifier genes in stem cells in order to force their expression and trigger differentiation into the lineage governed by said transgenes. Such strategy results in genetically modified cells carrying transgenes, whose controlled expression could be lost over time (mostly caused by progressive epigenetic silencing) and requiring the use of inducer agents like antibiotics and chemicals, potentially detrimental to human health and which are banned by several food agencies throughout the world. As it will be explained below the OSCs of the invention are devoid of any transgene insertion which is particularly advantageous in terms of genetic stability and safety of use in the food industry.

The step of processing the in vitro differentiated non-human animal cells which originate from at least one OSC can comprise harvesting said cells, optionally washing (or rinsing) the cells, mixing said cells with other food ingredients and/or to provide a foodstuff in a usual consumption form. Harvesting can be done by any method known in the art to recover cells from a cell culture suspension, as e.g. centrifugation, filtration or precipitation (i.e. flocculation, sedimentation or decantation), or a combination thereof, depending on, e.g., the volume and conditions of cell culture, specificities of the cells, the contemplated use of the harvested cells. For example, cell precipitation may be performed by adding to cell suspension calcium salt. The calcium salt may be selected from the group consisting of, but not limited to, calcium chloride, calcium acetate, calcium carbonate, calcium citrate, calcium gluconate, calcium lactate, calcium gluconolactate, calcium phosphate. Preferably, calcium chloride is used. The final concentration of the calcium chloride is in the range from 10 to 500 mg/L, preferably from 50 to 300 mg/L, more preferably is 50 mg/L. In another example, provided that culture volume is compatible, cell cultures can be centrifuged at a convenient speed depending on, e.g. if viable cells are required for the next step of processing or not, or on the desired compaction of the cells to be processed later on. Optionally, harvested cells can be washed or rinsed, for example with fresh media or saline solution then recentrifuged. Any other mean suitable to lower or to eliminate traces of culture medium compounds can be used alternatively or additionally (e.g. filtration, dialysis, precipitation . . . ).

Preferably said usual consumption form mimics visual appearance and/or also organoleptic properties of a conventional meat or meat-based product (i.e., comprising meat from bred animals and not from cultured cells). In that regard, said foodstuff can be based on a mix of several OSCs as described herein, or a mix of differentiated cells from these OSCs, at relative ratios that mimic particular tissues from fed animals. For example, said foodstuff can comprise a particular mix of adipocytes, skeletal muscle cells, skin (keratinocytes), nervous and/or cartilaginous cells at relative ratios corresponding to that of particular cuts of e.g., beef or poultry. Hence, preferably, the foodstuff according to the invention comprises OSCs, or differentiated cells from these OSCs and/or extracts thereof; in other words, cells can be intact undifferentiated and/or differentiated OSCs; and/or disrupted undifferentiated and/or differentiated OSCs.

The processing step can comprise the admixing of at least 1% in weight, with respect to a total weight of the foodstuff, of cultivated OSCs, or differentiated cells from these OSCs and/or cells extracts thereof. Preferably, at least 2% in weight, with respect to a total weight of the foodstuff, of cultivated OSCs, or differentiated cells from these OSCs and/or cells extracts thereof are mixed to other ingredients. More preferably, at least 5% with respect to a total weight of the foodstuff, of cultivated OSCs, or differentiated cells from these OSCs and/or cells extracts thereof are mixed to other ingredients. Even more preferably, at least 10% in weight with respect to a total weight of the foodstuff, of cultivated OSCs, or differentiated cells from these OSCs and/or cells extracts thereof are mixed to other ingredients. Said weight being preferably a weight of the wet foodstuff.

Preferably, 99% or less in weight with respect to a total weight of the foodstuff, of cultivated OSCs, or differentiated cells from these OSCs and/or cells extracts thereof are mixed to other ingredients during the processing step. More preferably, 95% or less in weight with respect to a total weight of the foodstuff, of OSCs, or differentiated cells from these OSCs and/or cells extracts thereof are mixed to other ingredients during the processing step. Even more preferably, 80% or less in weight of OSCs, or differentiated cells from these OSCs and/or cells extracts thereof are mixed to other ingredients during the processing step. The weight being preferably a weight of the wet foodstuff.

For example, the foodstuff comprises from 1% to 100% in weight of cultivated OSCs, or differentiated OSCs and/or cells extracts thereof, preferably from 1% to 99% in weight, even from 2% to 95% in weight, from 5% to 90% in weight, preferably from 10% to 80% in weight, more preferably from 20% to 70% in weight, or even more preferably from 30% to 60% in weight with respect to the total wet weight of the foodstuff.

Preferably, cells incorporated in the foodstuff result from the differentiation of OSCs of the invention; said cells can be at any stage of differentiation, i.e. at an intermediate stage of differentiation or at a late stage of differentiation. Cells at an intermediate stage of differentiation are cells for which at least one further differentiation step is possible. Cells at an intermediate stage of differentiation as illustrated in FIG. 1 can correspond to, for example, cells of the MD, ED, ectoderm type, cells from the ectodermal neural plate, neural crest, neural tube or epidermis, precursor cells of the ED lineage as foregut, midgut, hindgut, urogenital tract precursor cells, mesenchyme type cells, mesothelium type cells, non-epithelial blood cells, coelomocytes, intermediate MD type cells, chord type cells, paraxial MD type cells, or lateral plate MD type cells. Cells at a late stage of differentiation can be cells of any type as listed in FIG. 1.

In a particular embodiment, the processing step can comprise the admixing at least 1% in weight of hepatocytes, preferably at least 2% in weight of hepatocytes, more preferably at least 5% in weight of hepatocytes, even more preferably at least 10% in weight of hepatocytes with respect to the total wet weight of foodstuff, said hepatocytes originate from an OSC as exposed above. Preferably, the processing step can comprise the admixing of 99% or less in weight of hepatocytes, more preferably 95% or less in weight of hepatocytes, even more preferably 90% or less in weight of hepatocytes with respect to the total wet weight of the foodstuff, said hepatocytes originate from an OSC as exposed above. Said processing step can include the admixing of food ingredients and cooking steps, which results in a foie gras like duck liver pâté as described in the example section.

In a particular embodiment, the processing step can comprise admixing of at least 1% in weight of myocytes (such as skeletal muscle cells, cardiomyocytes, smooth muscle cells) resulting from differentiation of at least one OSC as described above, preferably the admixing of at least 2% in weight of myocytes, more preferably at least 5% in weight of myocytes, even more preferably at least 10% in weight of myocytes with respect to the total wet weight of the foodstuff. Preferably, the resulting foodstuff comprises 99% or less in weight of myocytes, more preferably 95% or less in weight of myocytes, even more preferably 90% or less in weight of myocytes with respect to the total wet weight of the foodstuff. For example, the foodstuff comprises from 1% to 100% in weight of myocytes, preferably from 1% to 99% in weight of myocytes, more preferably from 2% to 95% in weight of myocytes, even more preferably from 5% to 90% in weight of myocytes with respect to the total wet weight of the foodstuff.

In another particular embodiment, the processing step can comprise admixing of at least 1% in weight of keratinocytes issuing from differentiation of at least one OSC as described above, preferably the admixing of at least 2% in weight of keratinocytes, more preferably at least 5% in weight of keratinocytes, even more preferably at least 10% in weight of keratinocytes with respect to the total wet weight of the foodstuff. Preferably, the resulting foodstuff comprises 99% or less in weight of keratinocytes, more preferably 95% or less in weight of keratinocytes, even more preferably 90% or less in weight of keratinocytes with respect to the total wet weight of the foodstuff. For example, the foodstuff comprises from 1% to 100% in weight of keratinocytes, preferably from 1% to 99% in weight of keratinocytes, more preferably from 2% to 95% in weight of keratinocytes, even more preferably from 5% to 90% in weight of keratinocytes with respect to the total wet weight of the foodstuff.

Cells of animal origin are the most suitable compounds to reach the organoleptic properties of conventional meat products obtained from slaughtered animals. Indeed, cells or proteins of vegetable or fungal origin are meat alternatives that necessitate far more transformation steps and additive ingredients to mimic conventional meat products (particularly their flavor) obtained from slaughtered animals, and often failed to satisfactory reproduce tasting experience of consumer eating animal conventional food. OSCs according to the invention fulfil this need because they are of animal origin and therefore represent the closest counterpart of animal tissues from slaughtered animals and can be used at a lower environmental footprint compared to conventional meat products, providing competitive yields, and presenting no foreign DNA. Without being limited to this embodiment, as mentioned above, the processing steps can comprise admixing cells of different differentiation lineage, in order to obtain a foodstuff of a cellular composition like a foodstuff incorporating ingredient originating from a slaughtered animal, thereby resulting in improved organoleptic experience for the consumer. For example to reach as far as possible the appearance, texture, and/or flavor of piece of beef meat, neuronal, skeletal muscle, smooth muscle, hematopoietic cells, adipocytes can be admixed together in proportions similar to those known in the art for the animal originating piece of meat. Also, cells of different differentiation lineage can be arranged under different specific layers or parts (as e.g. a layer of meat topped by a layer of keratinocytes and/or adipocytes) which are associated in the foodstuff, or mixed together in an homogenous mix.

Other food ingredients to be mixed with OSCs, or differentiated cells from these OSCs and/or extracts thereof can comprise at least: a seasoning, a flavoring agent, a texturizer, a colorant, a preservative, or any other food ingredient (plant material, edible plant fat etc. . . . ) or a combination thereof.

A seasoning can, for example, be selected from: salt; pepper; garlic or shallot; aromatic herbs and/or spices, including rosemary, sage, mint, oregano, parsley, thyme, bay leaf, cloves, basil, chives, marjoram, nutmeg, cardamom, chiles, cinnamon, fennel, fenugreek, ginger, saffron, vanilla and coriander; alcohol, including wine, spirituous, cognac, armagnac, port wine, pineau des Charentes, rum, whisky, calva, pommeau de Normandie, jurançon, sauterne, pacherenc; or any combination thereof.

An edible plant fat can be selected from plant oils commonly used for cooking such as: canola oil, castor oil, coconut oil, flaxseed oil, allanblackia oil, olive oil, sunflower oil, soybean oil, peanut oil, illipe oil, cottonseed oil, shea oil, palm oil, avocado oil, safflower oil, sesame oil, lemon oil, grapeseed oil, macadamia oil, almond oil, sal oil, kokum oil, or, mango oil, or a combination thereof.

A flavoring agent can, for example, be selected from: a flavor enhancer, a sweetener or any combination thereof.

A texturizer can, for example, be selected from: a bulking agent or a thickener, a desiccant, a curing agent or any combination thereof.

A preservative can, for example, be selected from: an antimicrobial agent, a pH modulator, or any combination thereof.

A colorant should be suitable to be used in food; It can, for example, be selected from natural colorants such as carotenes, tomato, beet, or a mixture thereof.

The processing step may be done by any means well known to the skilled in the art. Non limiting examples of such process step are the solidification, pressing, heating, drying, freeze-drying, freezing, boiling, cooking, smoking, irradiating, homogenizing, under pressure cooking, molding, dosing, canning, pasteurization, extruding and/or packaging said differentiated cells and/or extracting components (e.g., proteins) from these differentiated cells.

Processing step can also comprise the use of texturizing techniques such as wet-spinning, 3D printing, electro-spinning, extrusion, soaking, liquid spraying, dry spraying, spray drying, ink jet application etc, applied either to differentiated cells or any derivative or extract thereof (protein fraction, fat fraction, etc). Hence, these can be used to produce a final product that presents the desired consistency, texture and appearance. Advantageously said texturizing techniques can be used to provide complex foodstuffs, comprising cells of different types organised in 3 dimensions (under in layer, insert) or simply mixed together to more accurately reproduce the appearance of a piece of meat originating from a slaughtered animal, in the foodstuff. For example, use can be made from different inks, each ink comprising different types of differentiated OSCs (e.g. endothelial, adipocyte, skeletal muscle and/or keratinocytes), said OSCs being for example mixed with other food ingredients as explained above, to reproduce meat products.

In some instances, differentiated cells can also have been further cultured under specific conditions, in a medium enriched in particular components to provide improved foodstuffs beneficial to the health of human or animal diets. Such components can be, for example, and are not limited to, essential trace elements, minerals, co-vitamins, essential fatty acids, essential amino acids, enzymes, antioxidants, etc. . . . In other instances, differentiated cells can be cultured under specific conditions in order to produce a specific food product or specialty food product. In that respect, it is known in the art that culturing hepatocytes in a medium enriched in fatty acids like saturated palmitic acid and/or monounsaturated oleic acid induces steatosis in a dose dependent manner (Moravcová et al., 2015). Steatosis is the accumulation in hepatocytes of triglycerides and fatty acids which is observed in foie gras specialty food. Also in a particular embodiment, the method of the invention relates to a method of producing foie gras from hepatocytes originating from at least one OSC, wherein said hepatocyte is cultured under conditions that promote steatosis, e.g., in a medium enriched in fatty acids, in such a way that triglycerides and/or fatty acids accumulate in said hepatocytes.

Preferably, cells are processed to obtain a foodstuff under a form selected from the group consisting of fresh product, a dried product, a frozen product, a powder, a paste, an extrudate, a solid or a liquid, optionally a product which has been or adapted to be minced, cooked, done, rehydrated, pickled or smoked. The foodstuff product can be processed to be defined as a processed food product, for example as a soup, a sauce, a topping, a seasoning, a stew, a sausage, minced meat, a meatball, a nugget, a spread, a pâté, a puree, a drink, or shake, a surimi, a biscuit, dried granules, tablets, capsules, a powder.

In a particular embodiment said method comprises, prior to the step of processing in vitro differentiated non-human animal cells, a step of producing said in vitro differentiated non-human animal cells which comprises:
a step of amplifying at least one OSC inactivated for the expression of at least one lineage specifier gene,
optionally a step of culturing said amplified OSCs as embryoid bodies, or
optionally, a step of differentiating said OSCs towards a specific cell type.

It should be understood that OSCs are modified PSCs that retain their ability to divide indefinitely, which is of particular interest in the field of synthetic meat products which requires high yields of cells, which are not obtainable with differentiated cells that have lost or have a restricted ability to grow. OSCs are able to self-renew and, when the desired cell density is obtained, to differentiate upon exposure to the suitable signalling.

The step of amplifying at least one OSC can be done by culturing undifferentiated OSCs with any method well known from the skilled in the art. Culture media are commercially available and well known from the skilled in the art for mammalian and other vertebrate cells. Further exemplary media for growth of crustacean cells are found in WO2020/149791. Also, culture media for insect cells can be found in Rosello et al. (2013). For example, undifferentiated OSC expansion can be achieved in matrix-dependent surface-attached two-dimensional (2D) cultures using conventional dishes or flasks by multiplying culture dishes or using multi-layered flasks. Alternatively, OSCs can be adapted to growth in three-dimensional (3D) matrix-dependent cultures or in instrumented stirred tank bioreactors as “free-floating” suspension cultures or using microcarriers providing anchorage-dependent OSCs with a “floating surface” enlarging the available surface area (Serra et al., 2012; Kropp et al., 2017).

Alternatively, the amplification step can be implemented using any method well known from the skilled in the art, depending on the cell lineage to which the at least one OSC is limited and on the desired differentiated cell type. For example, it is currently possible to establish self-renewing, ED-committed stem cell lines as described by Cheng et al. (2012). This approach is also applicable to ED-restricted OSCs and scalable in 2D using multiple culture dishes or multi-layered flasks, or in 3D using matrix-dependent cultures or instrumented stirred tank bioreactors. Another applicable approach to ED-restricted or, e.g., hepatic-restricted OSCs is described in Akbari et al. (2019) which allows the long-term expansion (up to one year) of PSC-derived hepatic organoids. The approach of Akbari et al. can also be applied to MD-committed OSCs. Such MD progenitor cultures can also be further amplified in 2D or 3D culture conditions. Alternatively, MD-restricted or cardiac-restricted OSCs can be amplified in suspension 3D culture as taught by Chen et al. (2014), Kempf et al. (2016) or Vahdat et al. (2019). Similarly, large-scale expansion of human iPSC-derived skeletal muscle cells has been documented (Van der Wal et al., 2018) and could be successfully applied to MD-restricted or skeletal muscle-restricted OSCs. These methods are easily adaptable to allow the amplification of any foodstuff relevant cell types (e.g., fibroblasts, red blood, or adipocytes).

The optional step of generating embryoid bodies from said amplified OSCs comprises detaching cells from the support onto which they are growing and allow them to grow in 3D suspension cultures. Cell aggregates spontaneously differentiate into progeny of the three embryonic germ layers upon removal of the signalling molecules maintaining pluripotency in the growing media. The cellular composition and gene expression signature of embryoid bodies generated from wild type PSCs is commonly used as an assay to quantify their differentiation potential towards the three embryonic germ layers. OSCs as described above are particularly advantageous for EB generation, as they are expected to yield a more homogeneous mass of cultured differentiated cells (i.e., enriched into cells from specific lineages) under EB differentiation culture conditions, when compared to embryoid bodies derived from non-modified PSCs. Moreover, EB cultures can be efficiently scaled up in bioreactors. In a particular embodiment, mass of differentiated cells obtained from EBs made of OSCs as described above are enriched of at least 10%, at least 20%, at least 30%, at least 40% even more preferably at least 50% in specific cell lineage or type related to said OSCs. As an example, mass of cultured differentiated cells from an EB made of OSCs restricted to MD lineage will be enriched of at least 10%, at least 20%, at least 30%, at least 40% even more preferably at least 50% in cells of MD lineage in comparison with differentiated cells obtained from EB made of PSC non-biased in their differentiation potency. Also, in another example, mass of cultured differentiated cells from an EB made of OSCs restricted to ED lineage will be enriched of at least 10%, at least 20%, at least 30%, at least 40% even more preferably at least 50% in cells of ED lineage in comparison with differentiated cells obtained from EB made of PSC non-biased in their differentiation potency; in yet another example, mass of cultured differentiated cells from an EB made of OSCs restricted to NE lineage will be enriched of at least 10%, at least 20%, at least 30%, at least 40% even more preferably at least 50% in cells of NE lineage in comparison with differentiated cells obtained from EB made of PSC non-biased in their differentiation potency.

In some instances, a further step of cell differentiation can be applied through specific culture condition, patterned scaffold and/or incubation with differentiation factor in order to trigger or make more efficient differentiation or further differentiation step of the OSCs according to the invention, thereby enriching the cell mass in tissue/organ specific cells. Those steps are well known from the person skilled in the art and vary as a function of the desired cell type. In other instances, OSCs restricted to early lineages can be processed as such to produce a foodstuff.

In a particular embodiment, the method of the invention comprises a prior step of obtaining said at least one OSC by stably inactivating at least one lineage specifier gene in a PSC. Any self-renewing totipotent stem cells (TSCs), PSCs, or multipotent stem cells (MSCs) can be used to produce an OSC according to the invention. Exemplary non-limiting examples are primordial germ cells (PGCs), female germline stem cells (FGSCs), spermatogonial stem cells (SSCs), embryonic germ cells (EGCs), ESCs, iPSCs, ntESCs and multipotent stem cells. ESCs are particularly preferred to obtain OSCs from a vertebrate origin since they don't require any exogenous transcriptional manipulation through reprogramming. ESCs from oviparous vertebrates are more particularly preferred as BDM cells can be easily isolated from early embryos, to create pluripotent ESC lines. Avian ESCs are particularly preferred, and even more particularly, duck ESC lines in order to produce any of the OSCs and, then, foodstuff deriving therefrom.

In a more particular embodiment, said stable inactivation of the expression of at least one lineage specifier gene is obtained by:
disrupting the reading frame of the coding sequence of said gene, and/or
inactivating a CIS-regulatory element required for the expression of said gene,
inactivating a TRANS-regulatory element required for the expression of said gene.

Inactivation of the function of at least one lineage specifier gene is typically achieved through knocking out of said gene, by disrupting the reading frame of the coding sequence of said gene. Alternatively, inactivation of the expression of at least one lineage specifier gene is typically achieved through deleting a portion or the entire promoter and/or enhancer sequences required for the expression of said gene. For the method of production of foodstuff of the invention, it is important that inactivation of at least one lineage specifier gene remains stable over the amplification steps of the cells. Generation of small insertion or deletion (indels) disrupting the coding sequence (CDS) of the gene or in some instance large deletions removing a large portion or the whole said gene or the promoter and/or enhancer sequences controlling the expression of said gene, without the introduction of foreign genetic material, further in the absence of any selection means, is therefore particularly suitable. Also, where generating indels in the lineage specifier gene is not desirable because the gene participates in or controls downstream essential processes or even not possible because of structural or other specificities of the target gene, stably inactivating CIS-regulatory elements (enhancers, silencers, tissue-specific regulatory elements), or TRANS-regulatory elements regulating the expression the target genes (Transcription factors, microRNAs, long noncoding RNAs) is particularly advantageous.

Any genome editing technology that enables indel knock out as exposed above is suitable to generate OSCs to be used in the methods of the invention. Such genome editing technologies are well known from those skilled in the art (reviewed in, e.g., Bennett et al. 2020). Technologies based on so-called programmable nucleases that comprises, but are not limited to, meganucleases, zinc finger nucleases, Transcription activator-like effector nucleases (TALENs) or Clustered Regularly Interspaced Short Palindromic Repeats (CRISPR)/CRISPR-associated nucleases (as, e.g., Cas 9) are widely used nowadays and particularly suitable to generate OSCs to be used in the methods of the invention. Further, resulting in OSCs being devoid of foreign DNA as e.g., selection marker or integrated vector, do not necessitate selection means during culturing and are considered as safer GMO (which do not present a risk for dissemination of foreign DNA). Also, in a particular embodiment of the method of the invention, stably inactivating at least one lineage specifier gene comprises generating at least one Indel in said gene with a gene editing system, preferably selected from a programmable nuclease selected from CRISPR/Cas9, TALENs, zinc finger nucleases, engineered nucleases as, e.g., MAD7, and/or meganucleases, even more preferably CRISPR/Cas9.

In an embodiment of the method of the invention, the at least one OSC is inactivated for the expression of at least one NE (see, e.g., genes of tables 1), MED (see, e.g. genes of table 3), MD (see, e.g., genes of table 4) or ED (see, e.g., genes of table 2) lineage specifier gene or a combination thereof. Suitable OSCs are described in the previous section.

In a particular embodiment of the method of the invention, said OSC is inactivated for the expression of at least one NE lineage specifier gene and for the expression of at least one MD lineage specifier gene. Indeed, these OSCs are restricted to differentiate into cells of ED lineage, and therefore provide valuable in vitro differentiated cells of ED lineage as foodstuff compounds. Inventors discovered that these OSCs can further be specialized in their differentiation potential for producing a single foodstuff-relevant cell type by further introducing organ-specifier knockouts to further restrict their differentiation potential towards specific cell types (see “Oligopotent stem cells” part above and, e.g., genes of Table 2). Accordingly, in a more particular embodiment of the method of the invention said OSC is hepato-specific and is inactivated for the expression of at least one gene of a NE lineage specifier gene, for the expression at least one gene of a MD lineage specifier gene and for the expression of at least one gene that governs differentiation of ED cells towards non-hepatic progenitor cells (see “Oligopotent stem cells” part above and, e.g., non-hepatocyte related genes of Table 2).

In another particular embodiment, of the method of the invention, said OSC is inactivated for the expression of at least one NE lineage specifier gene and for the expression of at least one ED lineage specifier gene. These OSCs are restricted to differentiate into cells of MD lineage, and therefore provide valuable in vitro differentiated cells of MD lineage as foodstuff compounds. Said OSCs can further be specialized in their differentiation potential for producing a single foodstuff-relevant cell type by further introducing organ-specifier knockouts to further restrict their differentiation potential towards specific cell types. For example, in a very particular embodiment, said OSC limited to the MD lineage, is further inactivated for the expression of at least one gene that governs differentiation of MD cells towards non-cardiac progenitor cells (see “Oligopotent stem cells” part above and, e.g., Table 4), said OSC being therefore cardiac specific. In another very particular embodiment said OSC is skeletal muscle specific and is therefore inactivated for the expression of at least one NE lineage specifier gene, for the expression of at least one gene of ED lineage specifier gene and for the expression of at least one gene that governs differentiation of MD cells towards non-skeletal muscle progenitor cell (see “Oligopotent stem cells” part above and, e.g., non-skeletal muscle-related genes of Table 4). In a further particular embodiment, said OSC is adipocyte specific and is therefore inactivated for the expression of at least one NE lineage specifier gene, for the expression of at least one gene of ED lineage specifier gene and for the expression of at least one gene that governs differentiation of MD cells towards non-adipocyte progenitor cells (see “Oligopotent stem cells” part above and, e.g., non-adipocyte related genes of Table 4).

In particular embodiments of the method of the invention, said at least one OSC is any of those described in the previous section related to OSCs. In a particularly preferred embodiment, said at least one OSC derives from a duck ESC.

Accordingly, in another embodiment of the method of producing a foodstuff of the invention, the at least one OSC is selected from a skeletal muscle, cardiac, hepatocyte, fibroblast, keratinocyte, red blood, or adipocyte specific OSC, or a combination thereof. In a more particular embodiment said at least one OSC is a combination. Indeed, admixing different OSCs at any of the amplifying, culturing and/or differentiating steps of the method of the invention allow to obtain masses of differentiated cells made of intimately intricate different cell types which are closer to the cellular composition of organs which originate from living animals and therefore improve the foodstuff in regard to its similarity with conventional foodstuff and therefore organoleptic experience of the consumer. Of course, in the method of producing foodstuff according to the invention, differentiated cells can also be admixed during the processing step. Also, in an embodiment of the method of the invention the in vitro differentiated non-human animal cells are selected from muscle cells, skin, blood cells, fibroblasts, adipocytes, or hepatocytes or a mix thereof.

### Foodstuff

Foodstuffs produced through the implementation of the method of the invention constitute an alternative to the consumption of meat products obtained from living animals. They represent a sustainable solution facing the growing world population, raising of the living standard, the shortage of natural resources and also the environmental concern related to farming, more specifically to intensive farming. Further, populations of western societies are increasingly concerned with animal welfare and drawbacks of intensive farming.

Nonetheless, in vitro meat production is still facing scalability requirements and high production cost. The method of the invention allows a scalable production of differentiated cells at a cheaper cost and/or in a safer way than in vitro meat of the art. Accordingly, an object of the invention relates to a foodstuff obtainable through the method of the invention as exposed above in any of its embodiments.

An object of this invention therefore relates to a foodstuff comprising at least one non-human animal cell, wherein said at least one non-human animal cell is inactivated for the expression of at least one lineage specifier gene selected from the groups of NE (as orthologs of genes listed e.g. in table 1), MD (as orthologs of genes listed e.g. in table 4), ED (as orthologs of genes listed e.g. in table 2) or MED (as orthologs of genes listed e.g. in table 3) lineage specifiers genes or a mix thereof.

A particular object of the invention relates to a foodstuff comprising at least one non-human animal cell wherein said at least one non-human animal cell is inactivated for the expression of at least one NE lineage specifier gene. In a particular embodiment, said foodstuff comprises at least one non-human animal cell is inactivated for the expression of:
at least one MD lineage specifier gene, or
at least one ED lineage specifier gene.

The skilled in the art will know how to confer to said foodstuff usual consumption form which mimics visual appearance and/or also organoleptic properties of a conventional meat tissues (muscle or offal) or meat-based product. The foodstuff product can be processed to be incorporated in a processed food product, in particular a soup, a stew, a sausage, minced meat, cold cuts, a spread, a pâté, a puree, a surimi, a biscuit, dried granules, tablets, capsules, a powder, pasta, a pizza, a sandwich or nuggets.

Though it could be considered that, when aiming to mimic meat-based products obtained from conventionally bred animals, the OSC originating from the same animals are to be used. For example, it can be inferred that when aiming to produce a foie gras, OSCs originating from goose or duck are preferable. . . . In another embodiment, it can be inferred that when aiming to produce a beef filet, OSCs originating from cow are preferable. Anyway, the use of cells originating from a different species, family, order or class can be considered.

The present invention is further explained with the following non-limiting examples.

### EXAMPLES

The following abbreviations have been used:
qRT-PCR: quantitative Reverse Transcription-Polymerase Chain Reaction.
PL: Pluripotent.
NE: Neurectoderm.
MED: Mesendoderm.
MD: Mesoderm.
ED: Endoderm.
Indel: Insertion or deletion.
fs: frameshift.
if: in frame.
CRISPR/Cas9: Streptococcus pyogenes type II clustered regularly interspaced short palindromic repeat/CRISPR-associated system.
gRNA: guide RNA, composed of two RNA molecules: a crRNA providing Cas9 nuclease its target specificity through 20 nucleotides of homology to DNA target sequence (protospacer), and a tracrRNA serving as binding scaffold for Cas9.
sgRNA: synthetic gRNA composed of a single RNA molecule retaining crRNA and tracrRNA functions.
RNP: Ribonucleoprotein
ROCKi: Rho-Kinase inhibitor.
EBd: embryoid body differentiation.
Dd: directed differentiation.
dESC: duck embryonic stem cell
ntESC: nuclear transfer ESC.
OSC: Oligopotent Stem cell. A self-renewing stem cell with restricted differentiation potential.
EDMD OSC: a self-renewing OSC, which differentiation potential has been restricted to ED, MD lineages, and their derivatives.
NEMD OSC: a self-renewing OSC, which differentiation potential has been restricted to NE, MD lineages, and their derivatives.
NEED OSC: a self-renewing OSC, which differentiation potential has been restricted to NE, ED lineages, and their derivatives.
NE OSC: a self-renewing OSC, which differentiation potential has been restricted to NE lineages and their derivatives.
MD OSC: a self-renewing OSC, which differentiation potential has been restricted to MD lineages and their derivatives.
ED OSC: a self-renewing OSC, which differentiation potential has been restricted to ED lineages and their derivatives.
Keratinocyte OSC: a self-renewing NE OSC, which differentiation potential has been restricted to epidermal lineage.
Liver OSC: a self-renewing ED OSC, which differentiation potential has been restricted to liver lineage.
Endothelial OSC: a self-renewing MD OSC, which differentiation potential has been restricted to endothelial lineage.
Blood OSCs: a self-renewing MD OSC, which differentiation potential has been restricted to blood lineage.
Heart OSC: a self-renewing MD OSC, which differentiation potential has been restricted to heart lineage.
Muscle OSC: a self-renewing MD OSC, which differentiation potential has been restricted to skeletal muscle lineage.
Bone OSC: a self-renewing MD OSC, which differentiation potential has been restricted to bone lineage.
Cartilage OSC: a self-renewing MD OSC, which differentiation potential has been restricted to cartilage lineage.
Fat OSC: a self-renewing MD OSC, which differentiation potential has been restricted to adipose lineage.
RTº: room temperature
WT: Wild Type

### Example I: Generating Mammalian OSCs—Inactivating Target Lineage Specifier Genes in PSCs

### I. Generating Edmd, Nemd, Need, Ne, Ed, Md Oscs Through Inactivation of Early Ne, Med, Md and En Lineage Specifier Candidate Genes in Hipscs.

WTC-11 human iPSCs (Coriell GM25256) are commercially well characterized iPSCs generated through non-integrative reprogramming of healthy male skin fibroblasts using episomal vectors.

### A. Cell Quality Assay

Prior to gene editing, undifferentiated, and differentiated (EBs) WTC-11 iPSCs were controlled for their PL-, NE-, MED-, MD-, ED-specific gene expression profiles and transcription factor markers expression profile using primers specific to the genes listed in table 5.

B. CRISPR/Cas 9 Nuclease Kit and gRNA Screening

CRISPR/Cas9 is currently the most widely used programmable nuclease for introducing fs Indels in the coding sequence of a target gene thereby knocking out the target gene. It has proven to work both in vivo, and in cultured cells from multiple species including vertebrates, invertebrates and plants (Kim and Kim, 2014). Numerous commercial off the shelf CRISPRCas9 kits are available.

Delivery of CRISPR/Cas9 components into the target cell is usually achieved through Amaxa nucleofection (Lonza), electroporation or lipofection. For editing WTC-11 iPSCs Alt-RR crRNAs, tracrRNA and S.p. HiFi Cas9 Nuclease V3 from Integrated DNA Technologies (IDT) were used complexed with Lipofectamine™ CRISPRMAX™ Cas9 Transfection Reagent from Invitrogen.

gRNA Design

Genomic and coding sequences of lineage specifier genes are downloaded from web-based genome browsers (e.g., website: ensembl.org/, website: genome.ucsc.edu/, website: ncbi.nlm.nih.gov/genome) and saved in a sequence analysis software (e.g., MacVector, SnapGene). Whenever possible, isoforms, exons and functional domains of each transcription factor are identified and annotated. 200-300 nucleotides of exonic sequence directly upstream of the DNA-binding domain of the transcription factor are used as sequence a query in gRNA design tool (website: en.wikipedia.org/wiki/CRISPR/Cas_Tools). The 3 highest-ranking gRNAs per gene with highest on-target and lowest off-target predicted activity are selected for further functional testing in WTC-11 iPSCs.

gRNA Screen

To identify the most active gRNA targeting each gene, all gRNA/Cas9 ribonucleoprotein (RNP) complexes are transfected separately in WTC-11 iPSCs using Lipofectamine CRISPRMAX Transfection Reagent following CRISPRMAX protocol. 4-5 days post-transfection, cells are collected from each well and DNA is purified using DNeasy Blood & Tissue Kit (Qiagen). For each target, locus TIDE/ICE oligo pairs are designed for amplifying a 500-700 genomic sequence flanking the gRNA target sites. Locus-specific PCR amplicons are purified using NucleoSpin Gel and PCR Clean up columns (Macherey-Nagel). Each product is sent for Sanger sequencing (Genewiz) using an internal TIDE/ICE sequencing oligo. Sanger sequence ABI files are then analyzed using TIDE (website: tide.nki.nl/) or ICE synthego (website: synthego.com/products/bioinformatics/crispr-analysis) software allowing quantifying small indels occurring at the site of cut of each gRNA.

### C. Generation of EDMD, NEMD, NEED OSC Clonal Lines.

Transfection of gRNA/Cas9 RNP Complexes.

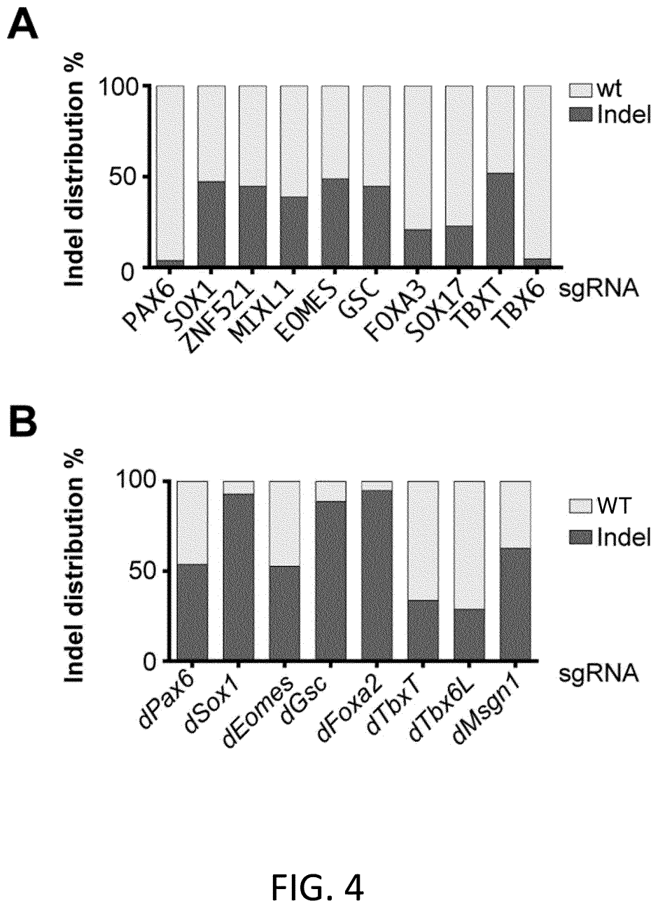

WTC-11 hiPSCs are transfected with the most effective gRNAs. WTC-11 cells are single-cell dissociated using TripleE Express (ThermoFisher), resuspended in E8 culture media (ThermoFisher) supplemented with 10 μM ROCKi (Y-27632, STEMCELL Technologies), and counted with a Countess automated cell counter (Life Technologies). 10 pools of 15*10⁵ cells are transfected with the corresponding RNP complexes in Vitronectin-coated (VTN-N, ThermoFisher) 12 well culture dishes using Lipofectamine CRISPRMAX following manufacturer's guidelines. 12-24 hours after transfection, the media is changed to E8 without ROCKi. Media is then changed daily until cells recover completely from transfection (4-5 days). As shown in FIG. 4A, it is found possible to generate indel in all the tested candidate lineage specifier genes.

Clonal Expansion of CRISPR-Edited hPSCs.

Each transfected pool is single cell dissociated using TripleE Express, resuspended in E8 supplemented with 10 μM ROCKi, counted with a Countess automated cell counter, and seeded at three different densities (50, 100 and 250 cells/cm2) in 100 mm VTN-coated culture dishes containing E8 supplemented with 10 μM ROCKi. 12-24 hours after transfection, media is changed to E8 without ROCKi. Media is then changed every second day until well defined colonies appear (7-10 days).

For each transfected gRNA, 96 colonies with a diameter greater than ˜500 μm are manually picked using a P200 pipette tip under an EVOS FL picking microscope (Life Technologies) and transferred to individual wells of a V-bottom 96-well dish. Using a multichannel pipette, colonies are manually disaggregated by pipetting up and down 10 times in the V-bottom dish followed by transfer to a VTN-coated 96 well culture dish containing E8 media. Media are changed every second day until well defined colonies appear (approximately 7 days).

At this stage, two replicas of each plate are generated by gently dissociating colonies using EDTA and replating each dissociated well into the corresponding wells of two VTN-coated 96 well replica culture dishes. 96 well replicas are cultured in E8 until 60-80% confluent, at which stage, one replica is frozen at −80° C. after EDTA dissociation and resuspension in PSC Cryopreservation Kit (ThermoFisher), while the other is used for genomic DNA extraction using DNeasy 96 Blood and Tissue Kit (Qiagen).

### Locus-Specific MiSeq Sequencing of CRISPR-Edited Clones.

Locus-specific Illumina MiSeq oligos are designed in order to amplify a 150-200 genomic region surrounding each gRNA cutting site. Each locus-specific MiSeq oligo pair is used for MiSeq PCR I amplification (15 cycles) of the 96 well plate containing the DNA edited with the gRNA targeting the corresponding locus using Herculase II Fusion DNA Polymerase (Agilent). PCR I products are diluted 10-fold in Nuclease-free H2O and 1 μl is used for MiSeq PCR II amplification (20 cycles) using oligos generating full length indexed (1-96) Illumina adapters flanking each locus-specific genomic amplicon. For each edit, the 96 barcoded PCRs are pooled, briefly migrated on 2% gel to remove primer dimers and purified using NucleoSpin Gel and PCR Clean up columns and eluted in Nuclease-free H2O. Each PCR pool is quantified using Qubit (ThermoFisher) and an equimolar amount of each PCR pool is mixed into a single tube and diluted in Nuclease-free H2O to generate a 10 nM final pooled library. Library quality-control and MiSeq run are performed by Genewiz (website: genewiz.com) using MiSeq Reagent Kit v3 (600 cycles) (Illumina).

### CRISPResso2 Analysis of MiSeq-Sequenced CRISPR-Edited Clones.

Trimmed MiSeq sequencing FastQ files corresponding to indexes 1 to 96 are retrieved from the Genewiz and sequences of the two alleles of each clone are identified and characterized using CRISPResso2 app (website: hub.docker.com/r/pinellolab/crispresso2/). Wild type, heterozygote, trans-heterozygote and homozygote clones carrying fs indels or fi indels are labelled for further amplification and storage.

### Amplification and Storage of CRISPR-Edited Clones.

96 well replica plates containing frozen clones are removed from −80° C., immediately thawed at 37° C. and centrifuged for 3 minutes at 300 g. Freezing media is removed by flicking the plates and immediately adding 100 μl E8 media containing RevitaCell Supplement 1× (ThermoFisher). Resuspended cells are transferred to VTN-coated 96 well culture dishes and grown overnight. 12-24 hours after plating, the medium is changed to E8 without RevitaCell Supplement. Medium is then changed daily until cells recover completely from freezing (7 days). Whenever possible, 3 (WT/WT) wild-type control clones, 3 (FS/WT) heterozygous clones and 3 (FS/FS) trans-heterozygous or homozygous clones are expanded in E8 into 24-well dishes and then in 6-well dishes using EDTA as a dissociation agent. IF/IF or IF/FS or IF/WT clones were also expanded when they were available. If possible, 3 to 6 cryovials of each line are cryopreserved in Nitrogen for banking and long-term storage.

### D. Generation of NE, ED, MD OSC Clonal Lines.

After having quantified the differentiation bias of EDMD, NEMD, NEED OSC clonal lines compared to wild type control lines (see below), one EDMD OSC line showing the highest differentiation bias towards EDMD, one NEMD OSC line showing the highest differentiation bias towards NEMD, and one NEED OSC line showing the highest differentiation bias towards NEED are selected.

### Generating NE OSCs.

Generating OSCs, which differentiation potential has been restricted to NE lineages and their derivatives, is performed using CRISPR/Cas9 technology as exposed above.

Three alternative approaches are applied:
1. transfecting 9 gRNAs targeting the 3 MED lineage specifiers in PSCs,
2. the best ED gRNA used for generating the best NEMD line is transfected in the best NEED OSC line,
3. the best MD gRNA used for generating the best NEED line is transfected in the best NEMD OSC line.

### Generating ED OSCs

Generating OSCs, whose differentiation potential has been restricted to ED lineages and their derivatives, is performed using CRISPR/Cas9 technology as exposed above.

Two alternative approaches are applied:
1. the best NE gRNA used for generating the best EDMD line is transfected in the best NEED OSC line,
2. the best MD gRNA used for generating the best NEED line is transfected in the best EDMD OSC line.

### Generating MD OSCs

Generating OSCs, which differentiation potential has been restricted to MD lineages and their derivatives, is performed using CRISPR/Cas9 technology as exposed above.

Two alternative approaches are applied:
1. the best NE gRNA used for generating the best EDMD line is transfected in the best NEMD OSC line,
2. the best ED gRNA used for generating the best NEMD line is transfected in the best EDMD OSC line.

The same editing and genotyping approaches are used as for generating EDMD, NEMD, NEED OSC clonal lines (see part C. above).

### II. Assessing the Differentiation Potential of PSCs and Lineage Restricted Oscs

### A. Pluripotency Assay.

This assay allows evaluating the pluripotency status of undifferentiated PSCs or EDMD, NEMD, NEED, NE, ED, MD OSCs by quantifying early PL, NE, MED, MD, ED gene expression and the percentages of early PL, NE, MD, ED cells in undifferentiated PSCs or OSCs.

PSCs or OSCs are grown in E8 media for two passages on VTN-coated 6 well culture dishes. Around 75% confluent wells are washed twice with PBS, dissociated with Accutase, and resuspended in PBS. Cells are counted with a Countess automated cell counter and 1*10⁶ cell aliquots pelleted. PBS is removed and cell pellets are resuspended either in 500 μl Trizol reagent (ThermoFisher) for TaqMan hPSC Scorecard Panel analysis (see section IV) or resuspended in 1 mL ice-cold FACS buffer (2% FBS in PBS) for flow cytometry analysis (see section E).

### B. EB Differentiation Assay.

This assay allows evaluating the differentiation potential of PSCs or EDMD, NEMD, NEED, NE, ED, MD OSCs using a low-stringency differentiation approach through quantifying early PL, NE, MED, MD, ED gene expression and the percentages of early PL, NE, MD, ED cells in EB-differentiated PSCs or OSCs.

### Exemplary Protocol 1 for EB Culture

PSCs or OSCs are grown in E8 media for two passages on VTN-coated 60 mm culture dishes. Around 80-85% confluent dishes are washed twice with PBS and treated for 5-10 minutes with Collagenase IV (ThermoFisher). Collagenase is then removed and cells are washed with 5 ml DMEM/F-12 (ThermoFisher). 3 ml EB medium (DMEM F-12 supplemented with GlutaMAX; KnockOut Serum replacement, MEM Non-essential Amino acids solution, and 2-mercaptoethanol) supplemented with 4 ng/ml bFGF (R&D Systems) is added to the cells. Colonies are carefully detached and collected using a 5 ml serological pipette. Colony suspension is transferred to a 15 ml conical tube and left to sediment for 5-7 minutes. Supernatant is carefully removed, and colonies are resuspended in 3 ml of EB medium with bFGF. These 3 ml are transferred to a non-TC treated 60 mm dish containing 2 ml EB medium with bFGF. The dish is placed in the incubator overnight. The following day, the content of the dish is transferred to a 15 ml conical tube and left to sediment for 5-10 minutes. The supernatant is removed and the EBs resuspended in 3 ml EB medium without bFGF (DO). The 3 ml EB suspension is transferred to a new 60 mm non-TC treated dish containing 2 ml EB medium without bFGF. After 7 days or 14 days in culture, EBs are harvested for analysis. After two washes with PBS, EBs are dissociated with Accutase, and resuspended in PBS. Cells are counted with a Countess automated cell counter and 1*10⁶ cell aliquots pelleted. PBS is removed and cell pellets are resuspended either in 500 μl Trizol reagent (ThermoFisher) for TaqMan hPSC Scorecard Panel analysis (see section D) or in 1 ml ice-cold FACS buffer (2% FBS in PBS) for flow cytometry analysis (see section E).

### Alternative Experimental Protocol for EB Culture

Undifferentiated hiPSCs (WTC-11) and OSCs are passaged and treated 24 h with 10 uM ROCK-inhibitor (Ri) Y-27632. Media is changed and the cells are left one more day with Essential 8 complete media (Gibco). The day of the experiment, cells are dissociated into single cells using PBS-EDTA 0.5 mM, filtered through a 37 μm cell strainer and counted. 2.5*10⁶ cells are transferred per well of 24-well Agrewell400 plate (STEMCELL Technologies) and incubated for 24 h with Essential 8 media containing 10 μM Ri. The next day, EBs are transferred to 6-well ultra-low attachment plates and incubated for 14 days with Essential 6 EB medium (Gibco). After 7 days or 14 days in culture, EBs are harvested for analysis. After two washes with PBS, EBs are dissociated with Accutase, and resuspended in PBS. Cells are counted with a Countess automated cell counter and 1*106 cell aliquots pelleted. PBS is removed and cell pellets are resuspended either in 500 μl Trizol reagent (ThermoFisher) for TaqMan hPSC Scorecard Panel (Thermofisher) analysis (see section D) or in 1 ml ice-cold FACS buffer (2% FBS in PBS) for flow cytometry analysis (see section E).

### C. Directed Differentiation Assays.

This assay allows evaluating late lineage differentiation potential of PSCs or EDMD, NEMD, NEED, NE, ED, MD OSCs using a high stringency directed differentiation approach through quantifying late NE, MD, ED gene expression and the percentages of late NE, MD, ED cells in directed-differentiated PSCs or OSCs.

To assay the capacity of NEED, NEMD and NE OSCs to generate late NE lineages, a protocol for directed differentiation towards TP63⁺ epidermal progenitors is used (Zhong et al., 2020).

PSCs or OSCs are passaged in E8 media on VTN-coated 6 well culture dishes. When ˜30% confluent (day 0=d0), E8 is switched to differentiation media (DMEM/F12, L-ascorbic acid, selenium, transferrin, insulin, 1× chemically defined lipid concentrate). From do to day 8 (d8), cells are cultured in differentiation medium with the following treatments: d0-6 (10 μM SB431542); d1-6 (5 μM CHIR99021); d1-8 (10 ng/ml BMP4 (R&D)); d4-8 (5 μM DAPT (Tocris 2634). At this stage (d8), cells are washed twice with PBS, dissociated with TrypLE Select and resuspended in PBS. Cells are counted with a Countess automated cell counter and 1*10⁶ cell aliquots pelleted. PBS is removed and cell pellets are resuspended in 1 mL ice-cold FACS buffer (2% FBS in PBS) for flow cytometry analysis (see section E below).

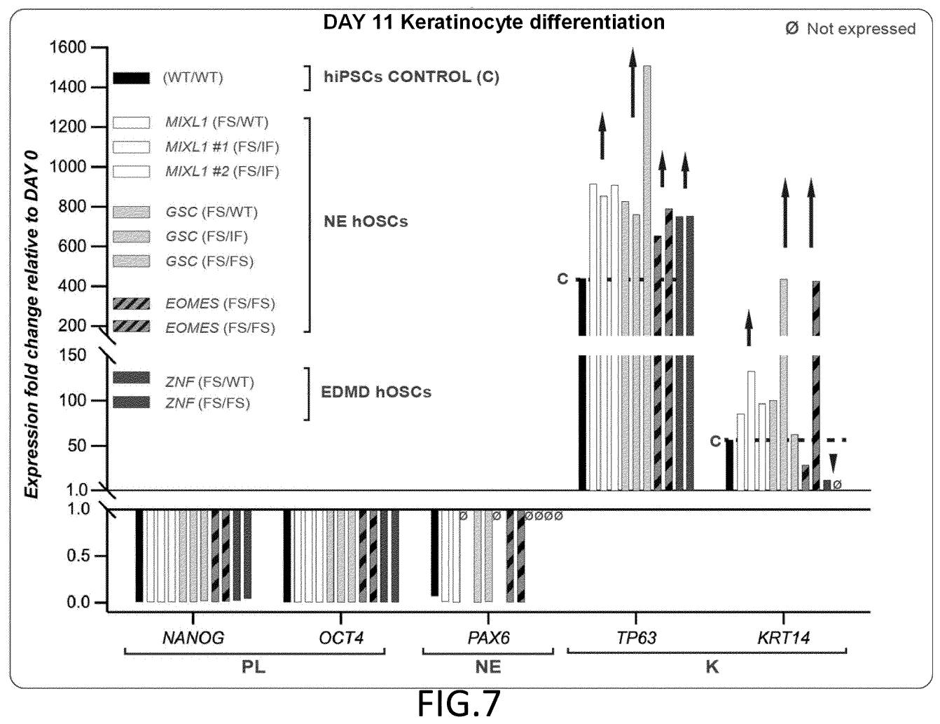

To assay the capacity of NE OSCs to generate keratinocytes NE OSCs are plated at 200000 cells/well in a 6-well plate, cultured for 3 days in E8 medium and then switched to differentiation medium (Defined Keratinocyte Serum Free Medium (DKSFM), 1 μM retinoic acid, 25 ng/mL BMP4) for 4 days. From day 4 to day 11, the media is changed every 2-3 days with DKSFM. From day 14 to day 25, the media was switched to CnT-07 and changed every 2-3 days. Data were analyzed according to the 2^{ΔΔCT} method as explained in section D below (on FIG. 7 are presented data from a D11 stage culture).

To assay the capacity of NEED, EDMD and ED OSCs to generate late ED lineages, a protocol for directed differentiation towards early PDX1⁺ pancreatic progenitors is used (Lee et al., 2019).

PSCs or OSCs are passaged at 65k-100k cells/cm² in E8 media on VTN-coated 6 well culture dishes. When around 80-90% confluent, pancreatic differentiation is initiated (do). Definitive endoderm (DE) is induced by treating with 100 ng/ml Activin A (PeproTech, 120-14E) for 3 days, 5 mM GSK-3 inhibitor, CHIR-99021 (Stemgent, 04-0004) for the first day, and 0.5 mM CHIR-99021 for the second day. Following treatment of 0.25 mM of L-Ascorbic acid (Sigma-Aldrich, A4544) and 50 ng/ml of FGF7 (R&D, 251-KG) for 2 days results in Foregut (FG) stage. PDX1⁺ early pancreatic progenitor cells are generated by adding 0.25 mM of L-Ascorbic acid, 50 ng/ml of FGF7, 250 nM of the hedgehog inhibitor, SANT-1 (Sigma, S4572), 1 mM of retinoic acid (Sigma, R2625), 100 nM of the BMP inhibitor, LDN-193189 (Stemgent, 04-0019), and 200 nM of PKC activator, TPB (EMD Millipore, 565740) for 2 days. At this stage (d7), cells are washed twice with PBS, dissociated with TrypLE Select and resuspended in PBS. Cells are counted with a Countess automated cell counter and 1×10⁶ cell aliquots pelleted. PBS is removed and cell pellets are resuspended in 1 ml ice-cold FACS buffer (2% FBS in PBS) for flow cytometry analysis (see section E below).

To assay the capacity of NEMD, EDMD and MD OSCs to generate late MD lineages, a protocol for directed differentiation towards ISL1⁺ cardiac progenitors was used (Balafan et al., 2020).

PSCs or OSCs are passaged in E8 media on VTN-coated 6 well culture dishes. When around 75% confluent, colonies are single cell dissociated using 0.5 mM EDTA and seeded at 2.4×104 cells/cm² on VTN-coated 6 well dishes in E8 Media supplemented with 10 μM ROCKi. After 24 h, media is changed to E8 without ROCKi and then changed daily. When 60-70% confluent (2-3 days) cells are treated with 6 μM CHIR99021 (Tocris Bioscience) in RPMI 1640 (Thermo Fisher Scientific) supplemented with B27-without insulin (Thermo Fisher Scientific) (do). After 24 h (d1) the medium containing CHIR99021 is changed to RPMI-B27 without insulin alone. After 48 h (d3) cells are treated with 5 μM IWP2 (Tocris Bioscience) diluted in RPMI-B27 without insulin. After 48 h (d5) medium is changed to freshly prepared RPMI-B27 without insulin. After 48 h (d7), cells are washed twice with PBS, dissociated with TrypLE Select and resuspended in PBS. Cells are counted with a Countess automated cell counter and 1*10⁶ cell aliquots pelleted. PBS is removed and cell pellets are resuspended in 1 mL ice-cold FACS buffer (2% FBS in PBS) for flow cytometry analysis (see section E).

D. Early PL, MED, NE, MD, ED Gene Expression Profiling Using TagMan hPSC Scorecard Panel.

For each TaqMan hPSC Scorecard Panel assay, 1*106 undifferentiated, EB-differentiated, directed-differentiated PSCs or OSCs resuspended in 500 μl Trizol (see sections A-C above) are used.

Total mRNA is purified through organic phase separation following manufacturer's guidelines. cDNA is generated by reverse transcription of 500 ng mRNA using the High-Capacity cDNA Reverse Transcription Kit following supplier's protocol (Applied Biosystem).

mRNA levels of PL-, NE-, MED-, MD-, ED-specific genes are then quantified using the TaqMan™ hPSC Scorecard™ Kit (Applied Biosystem). This predesigned TaqMan panel quantifies the expression of 85 lineage-specific genes including 9 PL, 6 MED, 26 ED, 22 MD and 22 NE genes (see Table 5 below. Human tri-lineage TaqMan hPSC Scorecard Panel). Based on the expression profile of these 85 genes, a differentiation score is attributed to each line using the software associated with the Taqman Scorecard Panel (website: apps.thermofisher.com/hPSCscorecard/home.html). This score reflects the differentiation potential of the line towards the 3 germ layers under undifferentiated or differentiation conditions.

TABLE 5
TARGET LINEAGE/lineage specific gene

PLURIPOTENT (9 GENES)
CXCL5; DNMT3B; HESX1; IDO1; LCK; NANOG; POU5F1; SOX2; TRIM22
MED (6 GENES)
FGF4; GDF3; NPPB; NR5A2; PTHLH; TBXT
ED (26 GENES)
AFP; CABP7; CDH20; CLDN1; CPLX2; ELAVL3; EOMES; FOXA1; FOXA2; FOXP2;
GATA4; GATA6; HHEX; HMP19; HNF1B; HNF4A; KLF5; LEFTY1; LEFTY2; NODAL;
PHOX2B; POU3F3; PRDM1; RXRG; SOX17; SST
MD (22 GENES)
ABCA4; ALOX15; BMP10; CDH5; CDX2; COLEC10; ESM1; FCN3; FOXF1; HAND1;
HAND2; HEY1; HOPX; IL6ST; NKX2.5; ODAM; PDGFRA; PLVAP; RGS4; SNAI2;
TBX3; TM4SF1
ECTODERM (22 GENES)
CDH9; COL2A1; DMBX1; DRD4; EN1; LMX1A; MAP2; MYO3B; NOS2;
NR2F1/NR2F2; NR2F2; OLFM3; PAPLN; PAX3; PAX6; POU4F1; PRKCA; SDC2;
SOX1; TRPM8; WNT1; ZBTB16
HOUSEKEEPING CONTROLS (5 GENES)
ACTB; GAPDH; RN18S1; TBP; UBC

For keratinocytes directed differentiation assays (section C above), the cDNAs are analyzed using TaqMan probes (Thermofisher, TaqMan™ hPSC Scorecard™ Kit, ref A 15872) for NANOG, OCT4, PAX6, TP63 and KRT14. GAPDH or ACTIN are amplified as internal standards. Data are analyzed according to the 2^{-ΔΔCT} method.

### E. Quantifying Early and Late PL, NE, MD and ED Cell Populations by Flow Cytometry.

1*10⁶ single cell aliquots resuspended in 1 ml ice-cold FACS buffer (2% FBS in PBS) are incubated 10 min at RT° with the appropriate LIVEDEAD Fixable Dead Cell Stain (Thermo Fisher Scientific) for discriminating dead cells from live cells. Cells are washed twice with FACS buffer. Fixation and permeabilization is performed using the BD Cytofix/Cytoperm Fixation/Permeabilization Kit (BD Biosciences) following manufacturer's guidelines. Fixed and permeabilized cells are collected in 300 μl of BD Perm/Wash buffer and aliquoted in six separate tubes mixed as follows: Tube 1 (50 μl BD Perm/Wash buffer); tube 2 (50 μl BD Perm/Wash buffer+Isotype control of conjugated antibody 1); tube 3 (50 μl BD Perm/Wash buffer+Isotype control of conjugated antibody 2); tube 4 (50 μl BD Perm/Wash buffer+Conjugated antibody 1); tube 5 (50 μl BD Perm/Wash buffer+Conjugated antibody 2); tube 6 (50 μl BD Perm/Wash buffer+Conjugated antibody 1+Conjugated antibody 2). Table 6 below (Human tri-lineage Flow Cytometry Panel) lists the antibodies used for these experiments. Each sample is incubated at RT° for 30 min. After incubation, samples are washed three times with 1 mL FACS buffer, resuspended in 300 μl FACS buffer, passed through a 40 μm cell strainer and stored on ice until flow cytometry analysis. For each sample, 20k events are captured on the MACSQuant X flow cytometer (Miltenyi Biotec) and analyzed with FlowJo X. Viability controls are performed with LIVE/DEAD™ Fixable Far Red Dead Cell Stain Kit, for 633 or 635 nm excitation (#L34974, ThermoFisher Scientific).

TABLE 6

| | | | |
|---|---|---|---|
| Cell labelling/control | Antibody | Catalog # | Supplier |

Pluripotency (PL) markers

| | | | | |
|---|---|---|---|---|
| UTF1+/NANOG+ | UTF1 | Alexa Fluor ® | 10276-202 | VWR |
| | | 350-conjugated | | |
| | | Rabbit IgG | | |
| | | Polyclonal | | |
| | | Antibody | | |
| | NANOG | PE-conjugated | IC1997P | R&D |
| | | Goat IgG | | Systems |
| | | Polyclonal | | |
| | | Antibody | | |
| | Isotype Ctl | Alexa Fluor ® | bs-0295P-A350 | Biossusa |
| | | 350-conjugated | | |
| | | Rabbit IgG | | |
| | | Isotype Control | | |
| | | PE-conjugated | IC108P | R&D |
| | | Goat IgG Isotype | | Systems |
| | | Control | | |

Early ED markers

| | | | | |
|---|---|---|---|---|
| SOX17+/FOXA2+ | SOX17 | Alexa Fluor ® | 562205 | BD |
| | | 488-conjugated | | Pharmingen |
| | | Mouse IgG1 κ | | |
| | | Monoclonal | | |
| | | Antibody | | |
| | FOXA2 | PE-conjugated | 561589 | BD |
| | | Mouse IgG1 κ | | Pharmingen |
| | | Monoclonal | | |
| | | Antibody | | |
| | Isotype Ctl | Alexa Fluor ® | 557782 | BD |
| | | 488-conjugated | | Pharmingen |
| | | Mouse IgG1 κ | | |
| | | Isotype Control | | |
| | | PE-conjugated | 555749 | BD |
| | | Mouse IgG1 κ | | Pharmingen |
| | | Isotype Control | | |

Early MD markers

| | | | | |
|---|---|---|---|---|
| TBXT+/TBX6+ | TBXT | Alexa Fluor ® | IC2085G | R&D |
| | | 488-conjugated | | Systems |
| | | Goat IgG | | |
| | | Polyclonal | | |
| | | Antibody | | |
| | TBX6 | Unconjugated | AF4744 | R&D |
| | | anti-TBX6 | | Systems |
| | | antibody | | |
| | | Polyclonal Goat | | |
| | | IgG | | |
| | Isotype Ctl | Alexa Fluor ® | IC108G | R&D |
| | | 488-conjugated | | Systems |
| | | Goat IgG Isotype | | |
| | | Control | | |

Early NE NE markers

| | | | | |
|---|---|---|---|---|
| NESTIN+/PAX6+ | NESTIN | PE-conjugated | IC1259P | R&D |
| | | Mouse IgG1 | | Systems |
| | | Monoclonal | | |
| | | Antibody | | |
| | PAX6 | Alexa Fluor ® | 562249 | BD |
| | | 647-conjugated | | Pharmingen |
| | | Mouse IgG2a κ | | |
| | | Monoclonal | | |
| | | Antibody | | |
| | Isotype Ctll | PE-conjugated | 555749 | BD |
| | | Mouse IgG1 κ | | Pharmingen |
| | | Isotype Control | | |
| | | Alexa Fluor ® | 558053 | BD |
| | | 647-conjugated | | Pharmingen |
| | | Mouse IgG2a κ | | |
| | | Isotype Control | | |

Late ED markers

| | | | | |
|---|---|---|---|---|
| PDX1+ identifies | PDX1 | Alexa Fluor ® | 562274 | BD |
| early pancreatic | | 488 Mouse IgG1 | | Pharmingen |
| progenitors | | κ Monoclonal | | |
| | | Antibody | | |
| | Isotype Ctll | Alexa Fluor ® | 557782 | BD |
| | | 488-conjugated | | Pharmingen |
| | | Mouse IgG1 κ | | |
| | | Isotype Control | | |

Late MD markers

| | | | | |
|---|---|---|---|---|
| ISL1+ identifies | ISL1 | Alexa Fluor ® | BOSSBS- | VWR |
| early cardiac | | 488-conjugated | 7532R-A488 | |
| progenitors | | Rabbit IgG | | |
| | | Polyclonal | | |
| | | Antibody | | |
| | Isotype Ctl | Alexa Fluor ® | 4340S | Cell |
| | | 488-conjugated | | Signalling |
| | | Rabbit IgG | | Technology |
| | | Isotype Control | | |

Early NE markers

| | | | | |
|---|---|---|---|---|
| TP63+ identifies | TP63 | Alexa Fluor ® | ab246727 | Abcam |
| early keratinocyte | | 488-conjugated | | |
| progenitors | | Rabbit IgG | | |
| | | Monoclonal | | |
| | | Antibody | | |
| | Isotype Ctl | Alexa Fluor ® | 4340S | Cell |
| | | 488-conjugated | | Signalling |
| | | Rabbit IgG | | Technology |
| | | Isotype Control | | |

Isotype Ctl: isotype control

### III. Generating Keratinocytes OSCs Through Inactivation of Late Ne, Ed, Md Lineage Specifier Candidate Genes in PSCs.

Keratinocyte OSCs are generated through gene editing in NE OSCs engineered from WTC-11 hiPSCs.

In order to restrict the differentiation potential of NE OSCs towards the keratinocyte (skin) lineage, three late neural lineage specifier genes (NEUROD2, SOX10, PAX3) are selected to be inactivated.

gRNA design, screen, transfection in NE OSCs, and generation of clonal lines is performed as described in part I above.

For each target gene keratinocyte lineage differentiation efficiencies are compared between unmodified PSCs, NE OSCs (WT/WT; FS/WT; FS/FS; FS/IF) and keratinocyte OSCs (WT/WT; FS/WT; FS/FS; FS/IF) using directed differentiation towards Tp63⁺ epidermal progenitors as described in part II above.

### IV. Results.

A. EB Differentiation Assay for WTC-11 hiPSCs.

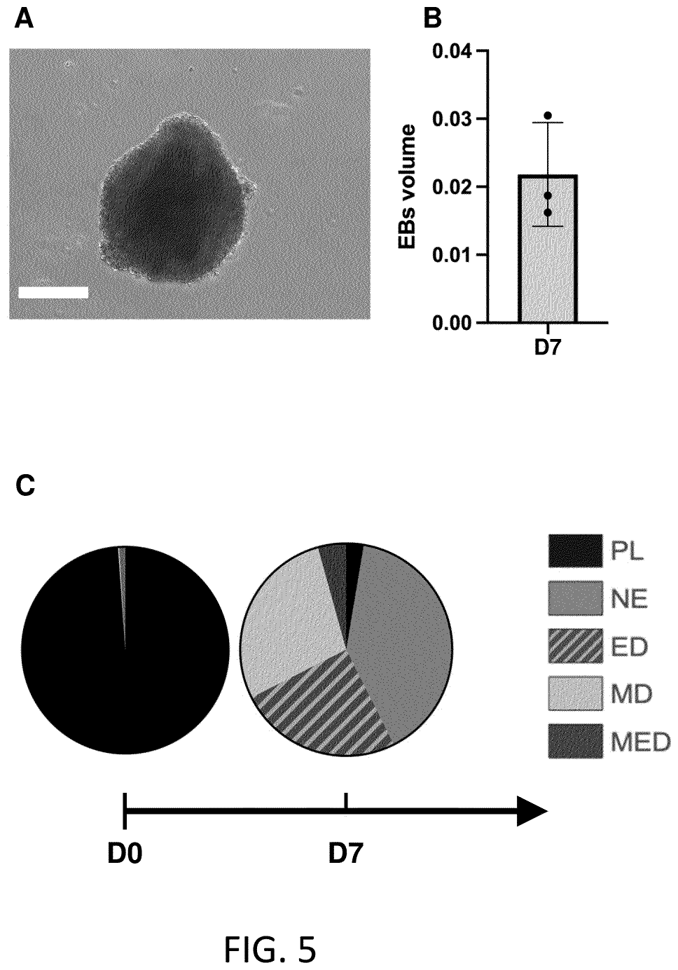

WTC-11 hiPSCs are able to form EBs in EB culture conditions, reaching 20 μm in diameter at day 7 (D7) of culture without bFGF. As expected, at day 0 (DO), around 98.85% of the contribution to gene expression is coming from pluripotency genes as determined using the TaqMan hPSC Scorecard Panel exposed above). A very small contribution (0.98%) comes from MED lineage genes and, even less (ranging from 0.01 to 0.04%) from ED, MD or NE genes (FIG. 5).

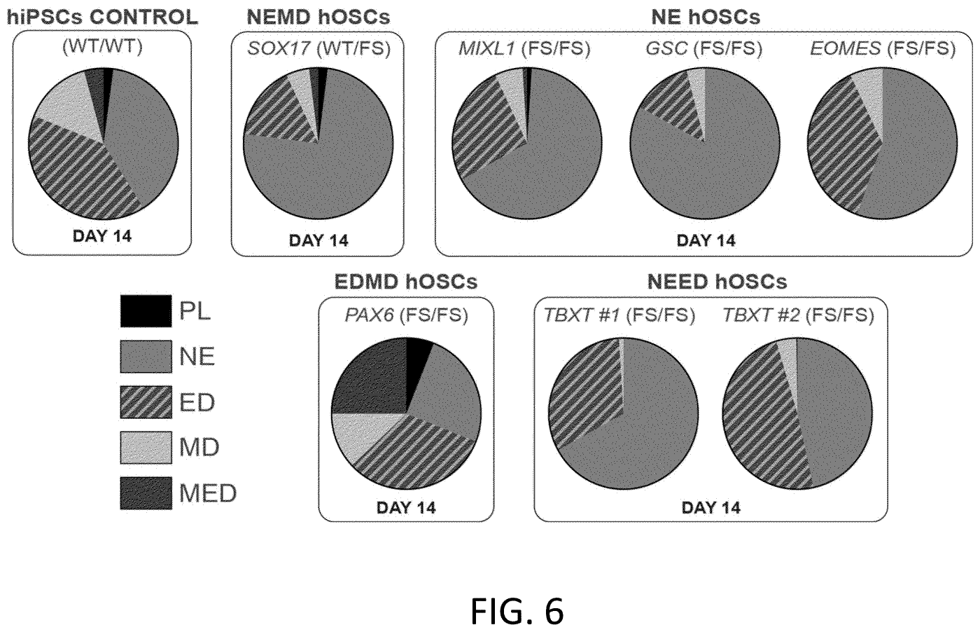

At D7 of culture, without bFGF in EB medium, hiPSCs cells efficiently differentiate into ED, NE and MD lineages: only 2.66% of the contribution to gene expression is coming from pluripotency markers, while the contribution to gene expression of ED, MD and NE genes reaches respectively 24.99%, 28.12% and 39.91%. At this stage, MED lineage genes only contribute to 4.31% of gene expression (FIG. 5 and summarized in table 7). At D14 of culture (FIG. 6), the contribution to gene expression of ED, MD and NE genes reaches respectively 38.00%, 14.00% and 37.00%.

TABLE 7
WTC-11 hiPSCs

| ko inactivated | Evolution of lineage representation in day 14 EBs | | | | |
|---|---|---|---|---|---|
| lineage | Pluripotent | | | | |
| specifier genes | cells | MED | MD | NE | ED |
| None | − | +/− | +++ | +++ | +++ |

−: strong loss;
+/−: slight increase;
+++: strong enrichment;

B. Early Lineage Restricted OSCs from hiPSCs.

As shown in table 8 for MD restricted OSCs, in table 9 for NE restricted OSCs, or in table 10 for ED restricted OSCs, or in FIG. 6, EB resulting from lineage restricted OSCs are significantly enriched in MD, NE, and ED early lineage cells in comparison to other types. Results are summarized in tables 8-10 below and also exemplified in FIG. 6.

TABLE 8
MD restricted OSCs

| | |
|---|---|
| ko inactivated | Enrichment of EBs in |

| lineage | Pluripotent | | | | |
|---|---|---|---|---|---|
| specifier genes | cells | MED | MD | NE | ED |
| PAX6 FOXA2 | − | +/− | +++ | + | + |
| PAX6 FOXA3 | − | +/− | +++ | + | + |
| PAX6 SOX17 | − | +/− | +++ | + | + |
| PAX6 FOXA1 | − | +/− | +++ | + | + |
| PAX6 HNF4 | − | +/− | +++ | + | + |
| SOX1 FOXA2 | − | +/− | +++ | + | + |
| SOX1 FOXA3 | − | +/− | +++ | + | + |
| SOX1 SOX17 | − | +/− | +++ | + | + |
| SOX1 FOXA1 | − | +/− | +++ | + | + |
| SOX1 HNF4 | − | +/− | +++ | + | + |
| ZNF521 FOXA2 | − | +/− | +++ | + | + |
| ZNF521 FOXA3 | − | +/− | +++ | + | + |
| ZNF521 SOX17 | − | +/− | +++ | + | + |
| ZNF521 FOXA1 | − | +/− | +++ | + | + |
| ZNF521 HNF4 | − | +/− | +++ | + | + |
| SOX2 FOXA2 | − | +/− | +++ | + | + |
| SOX2 FOXA3 | − | +/− | +++ | + | + |
| SOX2 SOX17 | − | +/− | +++ | + | + |
| SOX2 FOXA1 | − | +/− | +++ | + | + |
| SOX2 HNF4 | − | +/− | +++ | + | + |
| SOX3 FOXA2 | − | +/− | +++ | + | + |
| SOX3 FOXA3 | − | +/− | +++ | + | + |
| SOX3 SOX17 | − | +/− | +++ | + | + |
| SOX3 FOXA1 | − | +/− | +++ | + | + |
| ZIC1 HNF4 | − | +/− | +++ | + | + |
| ZIC1 FOXA2 | − | +/− | +++ | + | + |
| ZIC1 FOXA3 | − | +/− | +++ | + | + |
| ZIC1 SOX17 | − | +/− | +++ | + | + |
| ZIC1 FOXA1 | − | +/− | +++ | + | + |
| ZIC1 HNF4 | − | +/− | +++ | + | + |

−: strong loss;
+/−: slight increase;
+++: strong enrichment;
+: increase

TABLE 9
NE restricted OSCs

| ko inactivated | Enrichment of EBs in: | | | | |
|---|---|---|---|---|---|
| lineage | Pluripotent | | | | |
| specifier genes | cells | MED | MD | NE | ED |
| FOXA2 TBXT | − | +/− | + | +++ | + |
| FOXA2 TBX6 | − | +/− | + | +++ | + |
| FOXA2 MSGN1 | − | +/− | + | +++ | + |
| FOXA2 KLF6 | − | +/− | + | +++ | + |
| FOXA3 TBXT | − | +/− | + | +++ | + |
| FOXA3 TBX6 | − | +/− | + | +++ | + |
| FOXA3 MSGN1 | − | +/− | + | +++ | + |
| FOXA3 KLF6 | − | +/− | + | +++ | + |
| SOX17 TBXT | − | +/− | + | +++ | + |
| SOX17 TBX6 | − | +/− | + | +++ | + |
| SOX17 MSGN1 | − | +/− | + | +++ | + |
| SOX17 KLF6 | − | +/− | + | +++ | + |
| FOXA1 TBXT | − | +/− | + | +++ | + |
| FOXA1 TBX6 | − | +/− | + | +++ | + |
| FOXA1 MSGN1 | − | +/− | + | +++ | + |
| FOXA1 KLF6 | − | +/− | + | +++ | + |
| HNF4A TBXT | − | +/− | + | +++ | + |
| HNF4A TBX6 | − | +/− | + | +++ | + |
| HNF4A MSGN1 | − | +/− | + | +++ | + |
| HNF4A KLF6 | − | +/− | + | +++ | + |
| GSC | − | +/− | + | +++ | + |
| MIXL1 | − | +/− | + | +++ | + |
| EOMES | − | +/− | + | +++ | + |

−: strong loss;
+/−: slight increase;
+++: strong enrichment;
+: increase

TABLE 10
ED restricted OSCs

| ko inactivated | Enrichment of EBs in: | | | | |
|---|---|---|---|---|---|
| lineage | Pluripotent | | | | |
| specifier genes | cells | MED | MD | NE | ED |
| TBXT PAX6 | − | +/− | + | + | +++ |
| TBXT SOX1 | − | +/− | + | + | +++ |
| TBXT ZNF521 | − | +/− | + | + | +++ |
| TBXT SOX2 | − | +/− | + | + | +++ |
| TBXT SOX3 | − | +/− | + | + | +++ |
| TBXT ZIC1 | − | +/− | + | + | +++ |
| TBX6 PAX6 | − | +/− | + | + | +++ |
| TBX6 SOX1 | − | +/− | + | + | +++ |
| TBX6 ZNF521 | − | +/− | + | + | +++ |
| TBX6 SOX2 | − | +/− | + | + | +++ |
| TBX6 SOX3 | − | +/− | + | + | +++ |
| TBX6 ZIC1 | − | +/− | + | + | +++ |
| MSGN1 PAX6 | − | +/− | + | + | +++ |
| MSGN1 SOX1 | − | +/− | + | + | +++ |
| MSGN1 ZNF521 | − | +/− | + | + | +++ |
| MSGN1 SOX2 | − | +/− | + | + | +++ |
| MSGN1 SOX3 | − | +/− | + | + | +++ |
| MSGN1 ZIC1 | − | +/− | + | + | +++ |
| KLF6 PAX6 | − | +/− | + | + | +++ |
| KLF6 SOX1 | − | +/− | + | + | +++ |
| KLF6 ZNF521 | − | +/− | + | + | +++ |
| KLF6 SOX2 | − | +/− | + | + | +++ |
| KLF6 SOX3 | − | +/− | + | + | +++ |
| KLF6 ZIC1 | − | +/− | + | + | +++ |

−: strong loss;
+/−: slight increase;
+++: strong enrichment;
+: increase

Therefore, the ko by gene editing of at least one or a combination of lineage specifier genes in WTC-11 hiPSCs allows generating lineage restricted OSCs, thereby demonstrating that such approach is feasible for mammals. Indeed, As shown in FIG. 6, the inactivation of either MIXL1, GSC or EOMES results in OSCs developing embryoid bodies (EBs) which are significantly enriched in cells of NE (from 1.5- and up to 2.2-fold increase lineage while pluripotent cells and cells of ME, ED and MED lineage are significantly reduced in regard with control. As well, a significant bias in cell lineage composition of EBs is observed when a gene of the ED lineage is inactivated (e.g. SOX17, 2.0-fold increase of NE cells).

Inactivation of one gene governing differentiation into cells of early NE lineage (e.g. PAX6) results in an enrichment of more than 6.3-fold increase in cells of MED lineage, whereas inactivation of a gene governing differentiation into early MED (e.g. TBXT) results in producing embryoid bodies enriched in cells of NE lineage (up to 1.8 fold-increase) and ED lineage (up to 1.3-fold increase).

C. Directed Differentiation into Keratinocytes.

Results for directed differentiation of several clones OSCs inactivated for MED or early NE lineage specifier genes (FS/WT; FS/IF; FS/FS, FIG. 7) show, in regard to control a strong increase, in regard to wild type cells, of the expression of TP63 and/or KRT14 gene, usually considered as marker genes of keratinocytes.

### Example II: Generating OSCs from Duck ESCs

To show that engineering OSCs with restricted differentiation potential can be generally applied to non-mammalian species relevant for foodstuff production, duck OSCs are generated through gene editing in duck ESCs (dESCs).

dESCs are isolated in-house from freshly laid eggs and were quality controlled, prior to gene editing by qRT-PCR analysis of PL, NE, MED, MD, ED gene expression in undifferentiated and dESCs-differentiated EBs.

### I. Generating Duck OSCs Through Inactivation of Lineage Specifier Candidate Genes in Duck ESCs.

List of duck orthologs for the human genes used in this study is provided in Table 12 below.

TABLE 12

| | Access number | |
|---|---|---|
| Gene | NCBI-Gene | Species |
| FOXA2^{†} | 101795285 | Anas platyrhynchos |
| TBXT^{†} | 101795162 | |
| ACTB* | 101800437 | |
| OCT4^{†} | 100101567 | |
| ACTA2^{†} | 101793497 | |
| EN1^{†} | 101789433 | |
| DLX5^{†} | 101796922 | |
| LEF1^{†} | 101792052 | |
| PAX6 | 101799065 | |
| KRT14 (LOC101793676) | 101793676 | |
| TP63 | 101794454 | |
| MIXL1 | 113843121 | |
| GSC | 101791361 | |
| EOMES | 101804302 | |
| TBX6L | 101798203 | |

*control gene;
^{†}genes also used as marker of cell lineages (OCT4: pluripotent state; ACTA2, TBXT: mesoderm; EN1, LEF1, DLX5: neurectoderm; FOXA2: endoderm)

### A. Selection of Early Lineage Specifier Candidate Genes and Genome Engineering Platform.

As expected, the protein domains of the three MED duck orthologs and FOXA2, PAX6, SOX1 and TBX6 are well conserved between human and avian species. For generating duck genes knockouts, the approach described in Example I is used, aiming at generating fs Indels in the open reading frame of candidate genes through CRISPR/Cas9 editing in duck ESCs.

gRNA Design

Genomic and coding sequences of selected lineage specifier genes are downloaded from web-based genome browsers and saved in a sequence analysis software as described in Example I. 200-300 nucleotides of exonic sequence directly upstream of the DNA-binding domain of the transcription factor are used as query in CRISPOR gRNA design tool (website: crispor.tefor.net/). This gRNA design tool offers the option for designing and analyzing gRNAs over multiple invertebrate and vertebrate species including duck (Anas platyrhynchos). The 3 highest-ranking gRNAs per gene with highest on-target and lowest off-target predicted activity are selected for further functional testing in duck ESCs.

gRNA Screen

To identify the most active gRNA targeting each duck gene, the 9 duck MED gRNAs are transfected as described in Example I and genomic DNA from pools of cells transfected with a specific gRNA are analyzed. For each target, locus TIDE/ICE oligo pairs are designed for amplifying a 500-700 genomic sequence flanking the gRNA target sites. Locus-specific PCR amplicons are purified using NucleoSpin Gel and PCR Clean up columns (Macherey-Nagel). Each product is sent for Sanger sequencing (Genewiz) using an internal TIDE/ICE sequencing oligo. Sanger sequence ABI files are then analyzed using TIDE (website: tide.nki.nl/) or ICE synthego (website: synthego.com/products/bioinformatics/crispr-analysis) software allowing quantifying small indels occurring at the site of cut of each gRNA.

### Generation of Duck OSC Clonal Lines.

Transfection of gRNA/Cas9 RNP Complexes.

Same as Example I, except duck ESCs are cultured in dESC media (DMEM/F12 culture media (ThermoFisher) supplemented with Fetal Bovine Serum (Gibco) and LIF (Gibco) and passaged using 0.25% Trypsin-EDTA (Gibco). As shown in FIG. 4B, it has been possible to generate indels in all the tested genes, with efficiency even greater than for human WTC-11 cells clones.

Clonal Expansion of CRISPR-Edited dESCs.

Same as Example I, except duck ESCs are cultured with dESC media and passaged with 0.25% Trypsin-EDTA.

### Locus-Specific MiSeq Sequencing of CRISPR-Edited Clones.

Locus-specific duck Illumina MiSeq oligos are designed to amplify a 150-200 genomic region surrounding each gRNA cutting site. Each Locus-specific MiSeq oligo pair is used for MiSeq PCR I amplification (15 cycles) of the 96 well plate containing the DNA edited with the gRNA targeting the corresponding locus using Herculase II Fusion DNA Polymerase (Agilent). The rest of the approach is the same as Example I.

### CRISPResso2 Analysis of MiSeq-Sequenced CRISPR-Edited Clones.

CRISPResso2 analysis is performed as explained in Example I.

### Amplification and Storage of CRISPR-Edited Clones.

This step is performed as in Example I.

### B. Assessing the Differentiation Potential of Duck PSCs and OSCs.

### Pluripotency Assay

This assay allows evaluating the pluripotency status of undifferentiated duck ESCs or NE OSCs by quantifying early PL, NE, MED, MD, ED gene expression. Duck ESCs or NE OSCs are grown in dESC media for two passages on gelatin-coated 6 well culture dishes. Around 75% confluent wells are washed twice with PBS, dissociated with 0.25% Trypsin-EDTA, and resuspended in PBS. Cells are counted with a Countess automated cell counter and 1*10⁶ cell aliquots are harvested. PBS is removed and cell pellets are resuspended in 500 μl Trizol reagent (ThermoFisher) for qRT-PCR analysis. Primers amplifying amplicons of ˜150 bp, preferably in exon-exon junctions of selected genetic markers are designed as well known in the art. The Powertrack SYBR Green master mix (Applied Biosystems) is used to quantify the PCR associated signal using a real-time PCR machine (QuantStudio, ThermoFisher).

### EB Differentiation Assay

This assay allows evaluating the differentiation potential of duck ESCs or NE OSCs using a low-stringency differentiation approach through quantifying early PL, NE, MED, MD, ED gene expression. Protocol is roughly the same as in example I, briefly, undifferentiated duck ESCs and OSCs were cultivated until 80% confluency. Cells were enzymatically dissociated, counted and plated at 500000 cells/well in Ultra-Low Attachment (ULA) in ESCs medium (DMEM/F-12 without GlutaMAX, 10% FBS, 1.4% Glutamine, 1.2% Pyruvate, 1.2% NEAA, 0.2% 2-Mercaptoethanol, 10 ng/μl hLIF, 1.15 ng/μl IL6, 1.15 ng/μl IL6R, 1.15 ng/μl hSCF, 5.75 ng/μl iGF1) and put back in the incubator for 1 day. The next day, the aggregates were transferred in a 15 mL falcon tube and gravitationally sedimented for 5-10 min at room temperature. The supernatant was removed and the cells were resuspended using 6 mL of EB medium (DMEM/F-12 with GlutaMAX, 20% Knock-Out serum replacement, 1% MEM Non-Essential Amino Acids, 0.1% 2-Mercaptoethanol) and transferred to an Ultra-Low Attachment (ULA) 6-well dish. Media was changed every 2 days with fresh EB medium for 12 days.

After 12 days in culture, EBs are harvested for analysis. After two washes with PBS, EBs are dissociated with Accutase, and resuspended in PBS. Cells are counted with a Countess automated cell counter and 1*10⁶ cells aliquots were pelleted. PBS is removed and cells pellets are resuspended in 500 μl Trizol reagent (ThermoFisher) for analysis by qRT-PCR. Differentiation potential bias is determined by calculating the signal fold change between differentiated cells derived from control unmodified dESCs and duck NE OSCs.

### Alternative Protocol for EB Differentiation

AggreWell™400 24 well culture plates (StemCell technologies) are pre-treated with the Anti-Adherence Rinsing Solution (StemCell technologies) and bubbles present in the microwells are removed by centrifugation. Cells in adherent culture are dissociated for 5 minutes at room temperature TrypLE™ (Thermo Fisher) digestion and seeded into AggreWell™400 culture plates. A cell suspension of 6* 10⁵ cells (500 cells/microwell) is seeded in each well. Cells are incubated in these conditions for 24 hours at 37° C. and 5% CO2. After this incubation, small aggregates form. They are transferred to ULA plates (the content of one well is transferred to one 6-well plate well) and the media is changed to an EB media (composition described below). From this point on cells are incubated at 37° C., 125 rpm and 5% CO2. Media is changed every 2 days and the content of one P6 is collected for QCR analysis at every time point.

### Duck Keratinocyte Directed Differentiation

Undifferentiated duck ESCs and OSCs are plated at 10.000 cells/well in a 6-well plate, cultured for 3 days in ESCs medium (DMEM/F-12 without GlutaMAX, 10% FBS, 1.4% Glutamine, 1.2% Pyruvate, 1.2% NEAA, 0.2% 2-Mercaptoethanol, 10 ng/μl hLIF, 1.15 ng/μl IL6, 1.15 ng/μl IL6R, 1.15 ng/μl hSCF, 5.75 ng/μl iGF1) and then switched to differentiation medium (Defined Keratinocyte Serum Free Medium (DKSFM), 50 μM retinoic acid, 25 ng/mL BMP4) for 1 day. From day 1 to day 16, the media is changed every 2-3 days with Defined Keratinocyte Serum Free Medium (DKSFM).

total RNA is extracted from cell pellets generated at day 0 and day 16. After a step of reverse transcription, cDNAs are analyzed by qRT-PCR using specific primers spanning exon-exon junctions for each genetic marker, that were initially designed using the reference of genes listed in Table 12. SYBR green is used as a DNA binding fluorescent dye allowing the quantification of DNA molecules during the real time PCR.

### II. Results

A. EB Differentiation Assay for dOSCs.

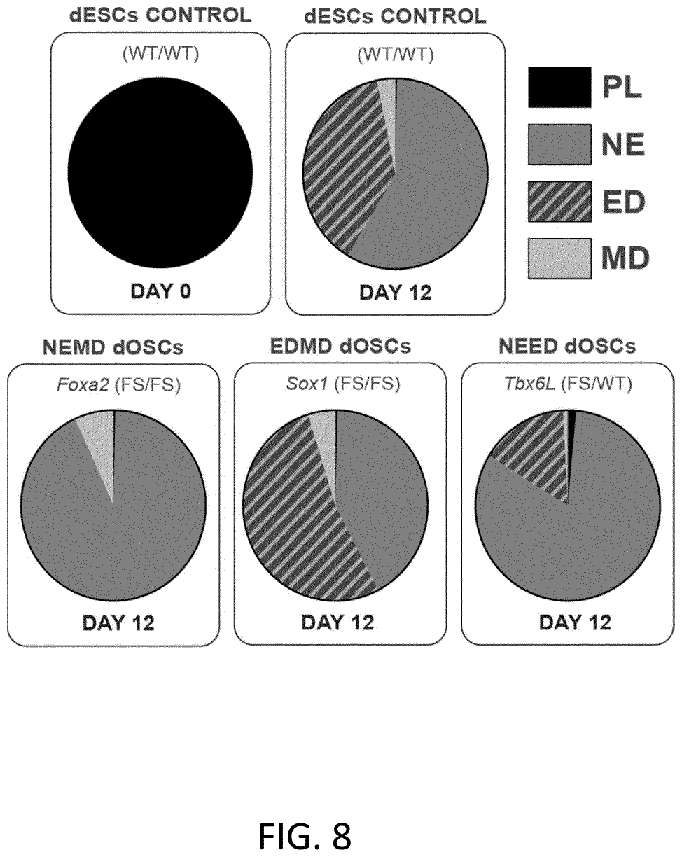

As for hiPSCs, for unmodified dESCs, after 7 days or 12 days of culture, ED, NE, and MD cells are found to constitute the majority of the cells of the EBs. Also, a significant enrichment is found in cells of NE lineage in EBs made of NE restricted-OSCs (inactivated for either duck gene ortholog of GSC, MIXL1 or, EOMES (not shown)). As shown in FIG. 8 inactivation of early ED, early NE or Early MD genes in duck ESC result in duck Embryoid bodies significantly enriched in non-inactivated lineage pathway cells. For example:
in EBs originating from NEMD and NEED dOSCs, NE cells represent most of the part of EB cells population and which is enriched up to 1.43-fold when compared to EBs obtained from non-modified dESCs.
in EBs originating from NEMD and EDMD dOSCs, MD cells population is also found increased by a factor of up to 2.26 when compared to EBs obtained from non-modified dESCs.
in EBs originating from EDMD dOSCs, ED cells population is also found increased by a factor of up to 1.65 when compared to EBs obtained from non-modified dESCs.

Conversely, a decrease in the population of cells corresponding to the inactivated lineage pathway is observed, for example,
in EBs originating from NEED dOSCs, a decrease up to 3.6-fold is found for ED or MD cell populations when compared to EBs obtained from non-modified dESCs,
In EBs originating from EDMD dOSCs a decrease of 1.55-fold in NE cells population is observed when compared to EBs obtained from non-modified dESCs.
   B. Directed Differentiation of NE OSCs into Keratinocytes.

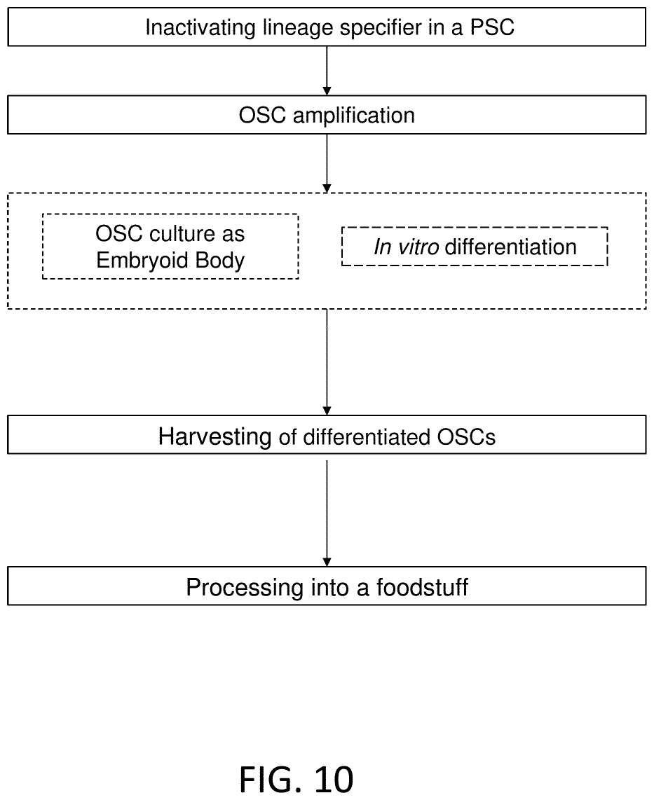

Two different double ko NE OSC clonal lines (Pax6 (FS/WT); Gsc (FS/FS) and Pax6 (FS/FS); Gsc (FS/FS)) are tested for their differentiation potential into keratinocytes using directed differentiation as exposed above (FIG. 10). In comparison with control, the two clones show a marked increase of the expression of Krt14, whose expression is considered in the art as a marker of keratinocytes. Conversely, said clones show a decrease in expression of NE marker genes as well as of PL genes.

### Example III: Processing Differentiated OSCs

Duck pâté

For preparation of duck pâthe, OSC-derived muscle cells and OSC-derived adipocyte cells are harvested separately by centrifugation at 300 g. Cell populations are combined (60% muscle cells and 40% adipose cells) and blended with appropriate amounts of onion, garlic, shallot, thyme, parsley and laurel. The mixture is then mixed with soy lecithin, flour, cognac, salt and pepper. The preparation is placed into a terrine dish full of water and baked for 5 to 90 minutes, preferably for 45 min between 60 to 200° C., preferably 160° C.

OSC-derived muscle cells and OSC-derived adipocytes are obtained from OSCs obtained as exposed above. After seeding in separate bioreactors and amplification for five days, said OSCs are then induced to differentiate and harvested separately by centrifugation at 300 g.

Duck Liver pâté

For producing a duck liver pâté, OSC-derived hepatocytes are obtained from OSC biased toward ED lineage. After seeding in a bioreactor and amplification at 37° C. with 5% CO2 for five days, said OSCs further differentiate into definitive ED cells and further mature hepatocytes using 10 ng/ml BMP4 and 20 ng/mL HGF cytokines. Said hepatocytes are then cultured under lipid over-loading conditions to achieve steatosis (RA Moravcová et al. 2015).

The obtained mature steatotic hepatocytes are then harvested and processed by centrifugation to remove the culture medium, optionally at least one washing/centrifugation cycle is applied on cells to remove culture medium. Supernatant is removed to keep the pellet of cells.

Harvested steatotic hepatocytes are then further processed with other food ingredients (Table 13, example of a duck liver pâté composition) to produce a duck liver pâté.

TABLE 13

| | % (in weight, with respect to a |
|---|---|
| Ingredients | total weight of the foodstuff) |
| Steatotic hepatocytes | 57 |
| Plant fat | 39 |
| Sugar | 1 |
| Salt | 0.9 |
| Texturizer | 0.8 |
| Armagnac | 0.6 |
| Spices | 0.4 |
| Pepper | 0.3 |
| TOTAL | 100 |

The steatotic hepatocytes are mixed with plant fat and all remaining food ingredients for 10 minutes using an industry standard high-shear mixing or a dispersion technology at 5000 RPM at a temperature of about 15° C.

The preparation is poured in a jar and then cooked at 70° C. (water bath) for 5-10 min and cooled down before storing it at 4° C. This results in a foie gras-like product.

### Beef Like Product

Beef embryonic stem cells are genetically engineered as exposed above to obtain OSCs biased toward MD lineage. After seeding in a bioreactor in a basal media suitable for beef OSCs and amplification at 37° C. with 5% CO2 for five days, said OSCs then differentiate into DE cells and further bovine skeletal muscle cells.

The cell biomass is then harvested and processed by centrifugation for 10 minutes at 1,500 g (at 4° C.) to remove cell debris and the culture medium, optionally at least one washing/centrifugation cycle is applied. Obtained cells are mixed with various food ingredients such as salt, pepper, sugar, texturizer, colorant, spices with an industry standard high-shear mixing or a dispersion technology at 5000 RPM, 15° C. for about 10 minutes. Those food ingredients are added to the cells in low quantities, between 0, 1 to 5% in weight with respect to a total weight of the intermediate cell-based preparation.

To further provide an appropriate appearance, texture and to mimic the mouthfeel of a conventional whole-cut piece of beef meat, an additional step of 3D printing is performed in order to post-process the final food product, using both an ink based of the intermediate cell-based preparation and an ink based of deodorized plant fat such as refined coconut oil. The printed foodstuff is then cooled down and packaged in a plastic bag under vacuum. The final packaged foodstuff is stored at 4° C. until further pre-consumption step such as pan-frying.

### REFERENCES

Akbari S, Sevinç G G, Ersoy N, Basak O, Kaplan K, Sevinç K, Ozel E, Sengun B, Enustun E, Ozcimen B, Bagriyanik A, Arslan N, Önder TT, Erdal E. Robust, Long-Term Culture of Endoderm-Derived Hepatic Organoids for Disease Modeling. Stem Cell Reports. 2019 Oct. 8; 13 (4): 627-641. doi: 10.1016/j.stemcr.2019.08.007. Epub 2019 Sep. 12. PMID: 31522975; PMCID: PMC6829764.
Balafan et al., A method for differentiating human induced pluripotent stem cells toward functional cardiomyocytes in 96-well microplates. Scientific Reports 2020.
Bennett E P, Petersen B L, Johansen I E, Niu Y, Yang Z, Chamberlain C A, Met Ö, Wandall HH, Frödin M. INDEL detection, the ‘Achilles heel’ of precise genome editing: a survey of methods for accurate profiling of gene editing induced indels. Nucleic Acids Res. 2020 Dec. 2; 48 (21): 11958-11981.
Campbell, K. H. S., McWhir, J., Ritchie, W. A. & Wilmut, I. Sheep cloned by nuclear transfer from a cultured cell line. Nature (1996).
Cheng X, Ying L, Lu L, Galvão A M, Mills J A, Lin H C, Kotton D N, Shen S S, Nostro M C, Choi J K, Weiss M J, French D L, Gadue P. Self-renewing endodermal progenitor lines generated from human pluripotent stem cells. Cell Stem Cell. 2012 Apr. 6; 10 (4): 371-84. doi: 10.1016/j.stem.2012.02.024. PMID: 22482503; PMCID: PMC3580854.
Evans, M. J. & Kaufman, M. H. Establishment in culture of pluripotential cells from mouse embryos. Nature (1981).
FAO (2011). World Livestock 2011-Livestock in food security. Rome, FAO website: fao.org/policy-support/tools-and-publications/resources-details/fr/c/1262785/
Kim and Kim., A guide to genome engineering with programmable nucleases. Nature Reviews Genetics 2014.
Christina Kropp, C., Massai, D., Zweigerdt, R, Progress and challenges in large-scale expansion of human pluripotent stem cells, Process Biochemistry, Volume 59, Part B, 2017, Pages 244-254, ISSN 1359-5113, (doi.org/10.1016/j.procbio.2016.09.032).
Lee et al., FOXA2 Is Required for Enhancer Priming during Pancreatic Differentiation. Cell Reports 2019.
Moravcová A, Červinková Z, Kučera O, Mezera V, Rychtrmoc D, Lotková H. The effect of oleic and palmitic acid on induction of steatosis and cytotoxicity on rat hepatocytes in primary culture. Physiol Res. 2015; 64 (Suppl 5): S627-36.
Post, M. J., Levenberg, S., Kaplan, D. L. et al. Scientific, sustainability and regulatory challenges of cultured meat. Nat Food 1, 403-415 (2020).
Rosselló RA, Chen C C, Dai R, Howard J T, Hochgeschwender U, Jarvis E D. Mammalian genes induce partially reprogrammed pluripotent stem cells in non-mammalian vertebrate and invertebrate species. Elife. 2013 Sep. 3; 2: e00036. doi: 10.7554/eLife.00036. PMID: 24015354; PMCID: PMC3762186.
Serra M, Brito C, Correia C, Alves P M. Process engineering of human pluripotent stem cells for clinical application. Trends Biotechnol. 2012 June; 30 (6): 350-9. doi: 10.1016/j.tibtech.2012.03.003. Epub 2012 Apr. 26. PMID: 22541338.
Takahashi, K. & Yamanaka, S. Induction of Pluripotent Stem Cells from Mouse Embryonic and Adult Fibroblast by Defined Factors. Cell (2006) doi: 10.1016/j.cell.2006.07.024.
Thomson J A, Itskovitz-Eldor J, Shapiro S S, Waknitz M A, Swiergiel J J, Marshall V S, Jones J M. Embryonic stem cell lines derived from human blastocysts. Science. 1998 Nov. 6; 282 (5391): 1145-7. doi: 10.1126/science.282.5391.1145.
United Nations, Department of Economic and Social Affairs, Population Division (2019). World Population Prospects 2019: Highlights (ST/ESA/SER.A/423). (website: population.un.org/wpp/Publications/Files/WPP2,019_Highlights.pdf).
van der Wal E, Herrero-Hernandez P, Wan R, Broeders M, In ‘t Groen S L M, van Gestel T J M, van IJcken W F J, Cheung T H, van der Ploeg A T, Schaaf G J, Pijnappel WWMP. Large-Scale Expansion of Human iPSC-Derived Skeletal Muscle Cells for Disease Modeling and Cell-Based Therapeutic Strategies. Stem Cell Reports. 2018 Jun. 5; 10 (6): 1975-1990. doi: 10.1016/j.stemcr.2018.04.002. Epub 2018 May 3. PMID: 29731431; PMCID: PMC5993675.
Zhong et al., Stagewise keratinocyte differentiation from human embryonic stem cells by defined signal transduction modulators. Int. J. Biol. Sci. 2020.

The invention claimed is:

1. A method for producing foodstuff comprising a step of processing in vitro differentiated non-human animal cells wherein said in vitro differentiated non-human animal cells originate from at least one oligopotent stem cell (OSC), said at least one OSC being inactivated for the expression of at least one lineage specifier gene.

2. The method according to claim 1 comprising, prior to the step of processing in vitro differentiated non-human animal cells, a step of producing said in vitro differentiated non-human animal cells which comprises a step of amplifying at least one OSC inactivated for the expression of at least one lineage specifier gene.

3. The method according to claim 2 comprising, prior to the step of amplifying the at least one OSC inactivated for the expression of at least one lineage specifier gene, a step of obtaining said at least one OSC by stably inactivating at least one lineage specifier gene in a Pluripotent Stem Cell (PSC) or multipotent or totipotent cell by generating at least one insertion and/or deletion with a gene editing system.

4. The method according to claim 3 wherein said PSC or multipotent or totipotent cell originates from a non-human vertebrate.

5. The method according to claim 3 wherein said PSC is selected from induced PSCs (iPSCs), embryonic stem cells (ESCs), or nuclear transfer ESCs (ntESCs) from non-human animal origin.

6. The method according to claim 1 wherein said OSC is inactivated for the expression of at least one neurectoderm (NE), mesoderm (MD), endoderm (ED) or mesendoderm (MED) lineage specifier gene selected from PAX6, SOX1, ZNF521, SOX2, SOX3, ZIC1, TBXT, TBX6, MSGN1, KLF6, FOXA1, FOXA2, FOXA3, SOX17, HNF4A, GSC, MIXL1 or EOMES or a combination thereof.

7. The method according to claim 6 wherein said OSC is inactivated for the expression of at least one NE lineage specifier gene and for the expression of at least one MD lineage specifier gene.

8. The method according to claim 6 wherein said OSC is hepato-specific and is inactivated for the expression of at least one gene of a NE lineage specifier gene, for the expression at least one gene of a MD lineage specifier gene and for the expression of at least one gene that governs differentiation of ED cells towards non-hepatic progenitor cells.

9. The method of claim 6 wherein said OSC is inactivated for the expression of at least one NE lineage specifier gene and for the expression of at least one ED lineage specifier gene.

10. The method of claim 6 wherein said OSC is skeletal muscle specific and is inactivated for the expression of at least one NE lineage specifier gene, for the expression of at least one gene of ED lineage specifier gene and for the expression of at least one gene that governs differentiation of MD cells towards non-skeletal muscle progenitor cell.

11. The method of claim 6 wherein said OSC is cardiac-specific and is inactivated for the expression of at least one NE lineage specifier gene, for the expression of at least one ED lineage specifier gene and for the expression of at least one gene that governs differentiation of MD cells towards non-cardiac progenitor cells.

12. The method of claim 6 wherein said OSC is adipocyte specific and is inactivated for the expression of at least one a NE lineage specifier gene, for the expression of at least one ED lineage specifier gene and for at least one gene that governs differentiation of MD cells towards non-adipocyte progenitor cells.

13. The method of claim 1, wherein the at least one OSC is a skeletal muscle, cardiac, hepatocyte, fibroblast, red blood, keratinocyte, or adipocyte specific OSC, or a combination thereof.

14. A non-human OSC characterized in that said OSC is inactivated for the expression of at least two lineage specifier genes selected from the groups of NE, MD, ED or MED lineage specifiers genes.

15. A foodstuff comprising at least one non-human animal cell wherein the expression of at least one lineage specifier gene, selected from the groups of NE, MD, ED or MED lineage specifiers genes, has been inactivated by generating at least one insertion and/or deletion with a gene editing system.

16. The method according to claim 2 wherein the step of producing said in vitro differentiated non-human animal cells further comprises a step of culturing said amplified OSCs as embryoid bodies.

17. The method according to claim 2 wherein the step of producing said in vitro differentiated non-human animal cells further comprises a step of differentiating said OSCs.
